SEQUENCE LISTING (2 sequences)

SEQ ID NO: 1 - DNA, 20 bp, Artificial Sequence
  Designed primer for performing real-time PCR
aactgttggccactatgagt

SEQ ID NO: 2 - DNA, 21 bp, Artificial Sequence
  Designed primer for performing real-time PCR
ccagcattacctgtaatctcg

(19) United States
(12) Patent Application Publication
(10) Pub. No.: US 2010/0279392 A1
(43) Pub. Date: Nov. 4, 2010

(54) Container for Liquid Reaction Mixture, Reaction-Promoting Device Using the Same and Method Therefor

(75) Inventors: Takashi Kodama, Hyogo (JP); Tomoko Kodama, Hyogo (JP); Kunio Tsuboi, Hyogo (JP)

Correspondence Address: Konomi Takeshita, Eight Penn Center, Suite 1300,, 1628 John F. Kennedy Blvd., Philadelphia, PA, 19103, US

(73) Assignee: TRUST MEDICAL CO., LTD., Hyogo (JP)

(21) Appl. No.: 12/601,469

(22) PCT Filed: May 23, 2008
(86) PCT No.: PCT/JP2008/059583

§ 371 Date: Mar. 31, 2010

(30) Foreign Application Priority Data
May 23, 2007 (JP) 2007-137299

(51) Int. Cl. C12M 1/38

(52) U.S. Cl. 435/286.1

(57) ABSTRACT

A container for a liquid reaction mixture whereby the liquid reaction mixture is brought into contact with a heater at a specified temperature and thus a nucleic amplification reaction is promoted, which has a substrate having a front surface and a back surface and being constructed so that either or both of these surfaces are maintained in facing a heater and wells that are formed in the surface direction of the substrate separately and independently from each other and each holds the above-described liquid reaction mixture in a liquid-tight sealed state, characterized in that; the above-described wells each comprises an opening formed in the above-described substrate and blocking members whereby the front surface side and back surface side of the opening are blocked, and at least the blocking member that blocks the surface in the side being in contact with the above-described heater has a part made of a stretchable film having a thickness of 10 to 300 μm.

### CROSS REFERENCE TO RELATED APPLICATIONS

This application claims priority under 35 U.S.C. 119 based upon Japanese Patent Application Serial No. 2007-137299, filed on May 23, 2007. The entire disclosures of the aforesaid application is incorporated herein by reference.

### FIELD OF THE INVENTION

This invention relates to a reaction container used in the biological fields, such as genetic engineering and enzyme engineering for promoting biological reactions which require temperature control, and to a reaction-promoting device using the same.

### BACKGROUND OF THE INVENTION

The PCR method (polymerase chain reaction), a revolutionary gene-analysis technology, was invented in 1983, and its use also permitted the Human Genome Project to be completed. However, the gene-analysis technology is still at a laboratory level in terms of practical speed and stability, and improvements thereof are needed for applications in clinical practice and production sites.

For example, although the PCR method mentioned above is a technology capable of amplifying a specific target DNA domain 100,000 fold or greater in a short period of time, it still requires tens of minutes and hardly meets the need for “several minutes” in clinical practice. Further from the standpoint of practical stability, it is noted that amplification may sometimes fail due to a cause such as fluctuation in heat transfer during a heating step for nucleic acid amplification. It may be permissible to repeat it, if failed, in the case of analysis for research, but it is an unavoidable problem when considering its practical application. Thus, the slow nucleic acid amplification speed and lack of amplification stability are stumbling blocks in the way of its widespread field application.

First, the problem of nucleic acid amplification speed is considered.

For example, generally, before the PCR method can be used in the medical field, there is required “a technology that stably amplifies the nucleic acid in a range of the 300 bps (to be explained below) needed in the emergency diagnostic field reproducibly in about 5 minutes or less.” Being able to stably perform amplification and identification of nucleic acid in about 5 minutes will make it possible for an emergency medicine practice under pressure of urgency to save many human lives, as well as to make a nucleic acid-based pathological diagnosis during an operation, and to prevent the spread of infection in the field of prevention of epidemics.

Notably herein, the nucleic acid-based diagnostic method consists of a “nucleic acid amplification step” and “an amplified nucleic acid identification step.” Nucleic acid amplification is a technology which multiplies a nucleic acid (DNA and RNA) several million fold via a thermal cycling reaction or an isothermal reaction, that is, an important technology which permits an analysis to be performed by multiplying the quantity thereof. The nucleic acid targeted for an analysis is a strand of several to several hundreds of thousands of bases, where for use in diagnosis or the like a fragment of the characteristic long nucleic acid is replicated for nucleic acid amplification. Although the length to be replicated is also dependent on the purpose, it is common to use a nucleic acid with a strand of 100 to 300 bases (called 100-300 bps).

Before the references for the prior art and the like are described, as an aid for comprehension, the classification on the basis of the type of nucleic acid amplification is shown in FIG. 1.

As shown in the Figure, the nucleic acid amplification method 1 is divided into “isothermal amplification method” 2 such as the Eiken Chemical's LAMP method and the like; and “thermal cycling amplification method” 3 with a high temperature thermal offset, such as PCR (polymerase chain reaction), LCR (ligase chain reaction), and the like.

The “isothermal amplification method” performs only an enzymatic reaction at constant temperature, and it is theoretically difficult to achieve a speed-up over the current technology. The “thermal cycling amplification method” promotes a nucleic acid amplification reaction with a thermal cycle applied to the reaction solution, whereby most of the required time is spent on raising and lowering the temperature, leaving much room for achieving a speed-up with improvement in efficiency of the heating and/or cooling. Moreover, from the standpoint of operational stability, each step being a thermal reaction, a stable contact between a heat source and a reaction container serves as a key to the stability. This prompts a detailed explanation of the thermal cycling amplification method below.

While “thermal cycling amplification methods” include the PCR method, the LCR method, and the others, an explanation will be given of the PCR method, a representative method among them.

Prepare in the PCR method, a reaction solution in which a primer that determines the replication origin and replication terminus regions, a template DNA, and a DNA extension enzyme (polymerase) have been mixed, thereby forming a double helix of the primer and the template DNA at a temperature of approximately 55° C. (hereafter “low temperature”). Then, allow the primer to be extended from the origin to the terminus regions by the polymerase at a temperature of approximately 75° C. (called “medium temperature”), thereby forming a replicated DNA. Further heat to around 95° C. (predetermined as “high temperature” hereafter) to separate the double strand of the replicated DNA into single DNA strands. Repetition of a thermal cycle of the low, middle, and high temperatures about 30 times theoretically permits the generation of approximately 2 raised to the 30^{th} power of copies of the replicated DNA.

The “thermal cycle amplification method “3 is classified into “stationary type” 4 and “flow type” 5 according to the way it is implemented. The flow type is one that moves the reaction solution held in a reaction container in a flow channel, thereby passing it through thermal regions at different temperatures and providing a thermal cycle. It is called a flow type because the reaction solution is allowed to flow in the container. On the other hand, the stationary type calls for moving, not the reaction solution but the reaction container, relative to the heat source to provide the reaction solution with a thermal cycle. It is called stationary in that the reaction solution is not moved in the reaction container.

Further, “stationary type “4 is divided into “open type” 6 and “sealed type” 7 based on whether or not the reaction vessel is open to the atmosphere. The thermal cycle amplification method includes a step for separating a double strand DNA with heat at 90° C. or higher, where the “open type” experiences a loss of water by evaporation, with a change in the liquid concentration, a serious shortcoming in stability. This makes the “open type” deficient in on-site practicality, suggesting the “sealed type” to be optimal in the clinical diagnostic field.

As mentioned above, clinical application demands a speed-up as well as stability. With attention focused on a speed-up, FIG. 2 shows the existing technology.

FIG. 2 tabulates commercial nucleic acid amplification devices A-F along with their heating systems and speed. Devices A-F, are compared in terms of heat source system and reaction time as shown as FIG. 3.

The PCR method is one said to be the global standard technology for nucleic acid amplification such that to say that the history of nucleic acid amplification is that of the PCR method is not an overstatement. A typical PCR device uses a system calling for placing the container on a “fixed heat source,” and raising and lowering the temperature of the source, which requires a given amount of time for changing the temperature up and down, a barrier to speeding up the operation. For example, A, although it is generally said to be a high-speed device, it still takes one hour.

On the other hand, PCR devices using a “gas heat source” took the lead in studies on speed-up based on the premise that use of a low heat-capacity “gas heat source” makes in effect negligible the time for raising and lowering the temperature of the heat source itself. This has resulted in devices B to D on the Table, advancing the high speed PCR's to levels of tens of minutes. Further, C and E speeded up close to 10 minutes, but not yet attaining a level of “5 minutes or less” that is truly demanded by the clinical diagnosis market. However, today, “low cost” and “ease of operation” are also sought in addition to “high speed” and “stability”, making it insufficient just to improve the heat source alone, requiring overall improvements of all elements of PCR, such as heat source, container, control, enzyme, and the like.

With attention focused on speed alone, there has been a recent proposal of a high-speed machine using high-pressure helium or high pressure carbon dioxide, as taught in U.S. Pat. No. 6,472,186 (reference 1). However, use of high-pressure helium or high-pressure carbon dioxide is highly problematic with respect to practicality in the emergency diagnostic field, not only for safety but also for ease of use and cost. In addition, there are devices using a similar “gas heat source”: device B (Japan Patent No. 3136129 official specification: reference 2) and device D (Tokuhyo [Published Japanese translation of a PCT application] No. 2000-511435 official specification: reference 3), both of which attempt to increase speed using a glass capillary, but fail to attain the target 5 minutes or less.

On the other hand, there is a device which is a fixed heat source—flow type (U.S. Pat. No. 6,780,617: reference 4). In this example a fixed heat source which is set at the three temperatures required in PCR is prepared and the reaction solution in a tubular container is moved back and forth with a plurality of pistons, thereby subjecting the reaction mixture to thermal cycles. This method, involving a complex structure, is not structurally expected to improve the speed.

Unlike a fixed heat source—flow type, a fixed heat source-stationary type moves, not the reaction solution within a reaction container, but the relative position of the reaction container to the heat source, thereby subjecting the reaction mixture to the thermal cycles. Among this type, device F (Tokuhyo No. 2004-504828 official specification: reference 5) calls for performing PCR by bringing the heat sources one after another into contact with the lower surface of a reaction chamber in the vicinity of a disk, except that the reference 5 gives no consideration of speed-up that the present invention aims at.

Unexamined Japanese application Publication No. 2005-115742 Specification (reference 6) discloses a PCR technology which comprises feeding a reaction solution to a flow channel constructed with a combination of a silicone rubber sheet having fine grooves, with plate glass, thereafter, moving it horizontally back and forth between 3 sets of fixed heat sources set at the temperatures required for PCR, and sandwiching the container, as it comes between a set of heat sources, on both surfaces thereof, thereby carrying out PCR. This technology is deficient in terms of reaction stability because the reaction chamber filled with the reaction mixtures is open to the atmosphere, making it impossible to prevent the escape of the vaporized steam. Furthermore, reference 6 indicates that a 30 cycle amplification of 200 bases takes 10 minutes, not yet meeting the market need for a speedup.

The references shown above are listed below.

Patent reference 1: U.S. Pat. No. 6,472,186 official specification.

Patent reference 2: Japan patent No. 3136129 official specification

Patent reference 3: Tokuhyo No. 2005-511435 official specification

Patent reference 4: U.S. Pat. No. 6,780,617 official specification

Patent reference 5: Tokuhyo No. No. 2004-504828 official specification Patent reference 6: Unexamined Japanese Patent Application Publication No. 2006-115742 official specification

As explained above, the conventional devices are so all variously deficient in terms of the conditions for practical nucleic acid amplification in clinical practice, such as speed-up and stability that none have yet reached commercialization.

Therefore, there is a strong demand for a device that can accelerate the reaction and is yet capable of withstanding the conditions of on-site use.

### SUMMARY OF THE INVENTION

This invention addresses the above situation, and according to the first main aspect, there is provided a container for a reaction solution for promoting a nucleic acid amplification reaction of the reaction solution by bringing it into contact with a heater at a predetermined temperature, the container comprising a base plate having front and back faces and being constructed so as to have either face thereof or both held facing the heater; and wells mounted on the base plate separately and independently from each other with each holding the reaction solution and being sealed liquid tight; wherein the well consists of an opening part provided on the base plate and a blocking member for blocking the base plate front and back face sides of the opening part, and wherein at least the blocking member which blocks the face of the side thereof in contact with the heater has a 400 μm thick stretchable film part.

According to such a construction, with the blocking member that blocks at least the face side of the well, which holds the reaction solution therein, in contact with the heater being a 10 to 400 μm thick stretchable film, heating by the heater expands the air within the sealed well and elongates the film, whereby the film is pressed against the heater. This raises the efficiency of heat transfer into the well, thereby accelerating the heating and cooling speeds of the reaction solution. This in turn permits shortening the thermal cycle given to the reaction solution. This enables realizing a 5 minutes-or-shorter thermal cycle.

Moreover, since the stretchable film will come in close contact with the heater, the stability in heat transfer increases, decreasing the possibility of the thermal cycle of reaction solution to fail.

Furthermore, as the number of wells present on the base plate increases, this will further enhance the practical effects, not only improving the heat transfer efficiency per well but also providing significant effect on heat transfer stability in imparting uniform heat to a large number of wells.

In addition, according to embodiment 1, the film portion is an olefin-based resin, a polyester based resin, a polyurethane resin, a silicone system resin, or a fluoropolymer based resin.

According to another embodiment 1, the blocking member containing the film portion is a film-like component adhered to the base plate.

According to yet another embodiment 1, the base plate comprises a stretchable resin material in which the opening constituting the well and the blocking member including film portion are integrated; and a rigid member and material which is fixed to the stretchable resin material and controls the expansion of the stretchable material in a direction of the face thereof.

According to yet another embodiment 1, the wells are constituted such that their internal pressure during the reaction becomes higher than the atmospheric pressure.

According to yet another embodiment 1, the nucleic acid amplification reaction is an isothermal nucleic acid amplification reaction.

According to yet another embodiment 1, the nucleic acid amplification reaction is a thermal cycle reaction including PCR (polymerase chain reaction) or LCR (ligase chain reaction).Herein, it is preferred for the base plate to be held so as to permit displacing its relative position with respect to the heater, thereby subjecting the reaction solution held in a well to a thermal cycle. Further in this case the base plate is to be held so as to permit displacing its relative position with respect to the heater in a rotating circumferential direction; and it is further preferred for the wells to be built around the center of rotation of the base plate.

The base plate may be held so as to permit displacing its relative position with the heater in a linear direction, wherein the wells may be built along the linear direction apart from each other.

According to yet another embodiment 1, the wells are built so as to block an optical interference between each well. In this case, it is preferred for the base plate to be at least partially made of a light-shielding material or colored to shield light. The base plate may be a type wherein either the front or back face of the base plate has a metal reflective film.

According to yet another embodiment 1, there is provided a container for a reaction solution, wherein the container is so constructed that after the reaction solution is introduced into an opening part of the well, adhering either one of the blocking members to the base plate seals the opening part of the well liquid-tight; wherein either a proximal part of the one of the blocking members of the interior wall of the opening part or the face of the blocking member that faces the opening part, or both are hydrophobic.

According to the second main aspect of the present invention, there is provided a reaction promoting device for promoting the nucleic acid amplification reaction of a reaction solution by having installed a heater capable of heating to a predetermined temperature and heating the reaction solution held in an independent well formed in a container for a reaction solution, wherein the well consists of an opening part built in a base plate and blocking members for blocking the base plate front and back face sides of the opening part, wherein at least the blocking member which blocks the face of a side in contact with the heater has a 10 to 400 μm thick stretchable film, and wherein the device is controlled to heat the interior of the well by heating with the heater whereby the expansion of the contents thereof brings the stretchable film into close contact with the heater.

According to the 3rd main aspect of the present invention, there is provided a reaction promoting method for promoting the nucleic acid amplification reaction of a reaction solution by heating, with a heater capable of heating to a predetermined temperature, the reaction solution held in an independent well formed in a container for a reaction solution, wherein the well consists of an opening part built in a base plate and a blocking members for blocking the base plate front face and back face sides of the opening part, wherein at least the blocking member which blocks the face of a side in contact with the heater has a 10 to 400 μm thick stretchable film, and wherein the method comprises the step of sealing the reaction solution in the independent well at a temperature not higher than a predetermined temperature and the step of heating the interior of the well with the heater to the predetermined temperature or higher whereby the expansion the expansion of the contents thereof brings the stretchable film into close contact with the heater.

According to the 4th aspect of the present invention, there is provided a container for a reaction solution wherein the container is so constructed that the base plate constituting the reaction container is provided with a liquid inlet aperture and a plurality of connecting paths connecting the liquid inlet aperture with each of the wells, whereby the reaction solution filled in the aperture is transported through the connecting path into the wells.

Preferably in this case, the plurality of connecting paths is constructed such that at least one of them is shallower than the others and the shallower connecting path is used as an air vent when the reaction solution is transferred to the well.

Moreover, the base plate may be a type wherein an air vent hole is formed therein and a connecting groove for connecting the air vent hole to the well is formed.

It is preferred for the stopper used for sealing the liquid inlet aperture or the air vent hole to have a groove formed in part of its seal face (side face part) so as to enable venting the air in the container to the atmosphere when the stopper is inserted thereinto, and permitting the sealing when the stopper is completely inserted.

The base plate may be constructed to be provided with an air venting groove-like aperture connected to the well, a seal liquid aperture filled to be with a seal liquid for allowing the seal liquid to flow, and a groove-like aperture that connects the seal liquid aperture, the air venting groove-like aperture, and the liquid inlet aperture such that after transport of the reaction solution from the liquid inlet aperture to an aperture-for-reaction, the seal liquid is transported thereto from the aperture filled with the seal liquid, thereby introducing the seal liquid into both the groove-like aperture at the liquid inlet aperture side of the aperture-for-reaction and the groove-like aperture at the air vent side.

In addition, it is preferred for the seal liquid in the above case to be a high viscosity grease-like liquid including silicone grease, or a UV curable resin, or a thermoset resin.

The above container for a reaction solution may be such that the wells are flow channel-shaped, one end of the flow channel-shaped well is a reaction solution inlet port, the other end is either open to the atmosphere or is maintained above atmospheric pressure, and that the internal pressure of the well during the reaction is made higher than atmospheric pressure by letting the reaction solution continuously flow through the reaction solution inlet port.

Moreover, the reaction may be nucleic acid amplification in which electrophoresis is performed following the nucleic acid amplification.

The flow channel-shaped well may comprise a part made to face a heater which is set at a temperature for causing reverse transcription of the RNA, a part made to then face a heater which is set at a temperature for deactivating the reverse transcriptase, and a part made to face thereafter a heater set at a temperature corresponding to nucleic acid amplification reaction.

According to the 5th main aspect of the present invention, there is provided a reaction promoting device for promoting a reaction of a reaction solution using a container for a reaction solution, wherein the container for a reaction solution is so constructed that electrodes are installed for applying an electrophoretic voltage at a total of two sites, one at the end of a flow channel—shaped well where the nucleic acid amplification has ended and one just proximal thereto; a new flow channel is installed that intersects with a part between the electrodes of the flow channel-shaped well; open ends are provided at the two ends of the new flow channel, and at the respective open ends thereof, electrophoresis electrodes are also deposed for applying an electrophoretic voltage; wherein the transfer of the liquid is stopped when the nucleic acid amplification reaction has progressed and the reaction solution has reached the end of the flow channel, which is followed by causing an electrophoretic gel to flow from the open end of the new flow channel until the gel appears at the other open end when the flow is stopped, applying a voltage across the end of the flow channel and a site just proximal thereto, and thereafter applying a voltage across the new flow channel.

According to embodiment 1, the container for a reaction solution comprises Peltier elements which sandwich the base plate and a transparent member, and is constructed to promote the nucleic acid amplification reaction of the reaction solution by controlling the Peltier elements at temperatures corresponding to those for the nucleic acid amplification reaction by a thermal cycle, wherein an optical measurement device is configured to illuminate the reaction solution through the transparent member and measure optical changes that have occurred.

According to the 6th main aspect of the present invention, there is provided a reaction container wherein in the container, a concave part is formed into the flat plate part of the container having a flat plate part; a reaction solution, which is an aqueous solution, is placed in the concave part; thereafter the concave part is sealed, thereby sealing off the reaction solution; and a fixed heat source set at a temperature corresponding to the temperature needed for the reaction is brought into contact with the flat plate part thereby performing nucleic acid amplification reaction; and wherein at least one circumferential lip is formed on an upper end face of the concave part; and both the circumferential lip part and the lower face of the seal member are made hydrophobic, thereby preventing the reaction solution from entering the sealed part due to the reaction solution's surface tension and thus allowing the nucleic acid amplification reaction to be stably performed.

According to embodiment 1, the nucleic acid amplification reaction is a nucleic acid amplification reaction which requires thermal cycles, such as PCR (polymerase chain reaction) or LCR (ligase chain reaction). In this case, the nucleic acid amplification reaction is preferably a reaction performed in an emulsion state.

According to another embodiment 1, the nucleic acid amplification reaction is an isothermal nucleic acid amplification reaction, such as the LAMP method or the like.

According to another embodiment 1, the container is characterized in that in the container a reverse transcription reaction is performed in a aperture-for-reaction followed by heating to a temperature for deactivating the reverse transcriptase thereby deactivating the reverse transcriptase and by performing nucleic acid amplification.

According to yet another embodiment 1, the container is characterized in that the container has a center of rotation; and a flat plate part having an aperture-for-reaction is on the circumference thereof, wherein the container comprises a plurality of apertures for reaction built separately and independently apart from each other across the radial and circumferential directions with respect to the center of rotation.

According to yet another embodiment 1, the container is characterized in that the container has a center of rotation; a flat part with apertures for reaction is circumferentially shaped; and the apertures for reaction are distributed across the entire face of the circumferentially shaped flat plate.

According to yet another embodiment 1, the container is characterized in that at least one side of the aperture-for-reaction of the container flat plate part is a 10-400 μm thick film.

According to yet another embodiment 1, the container is characterized in that either the container flat part is made from a light-shielding material or the wall face around the aperture-for-reaction is coated with a metal film, thereby shielding it from the effect of light from the adjacent apertures for reaction.

According to yet another embodiment 1, the container is characterized in that it comprises a metal film to reflect the light incident on the aperture-for-reaction of the container flat part.

According to the 7th main aspect of the present invention, there is provided a nucleic acid amplification device characterized in that in the device for performing a thermal cycle reaction using the reaction container, the device comprises a mechanism for rotating the reaction container, a plurality of fixed heat sources corresponding to thermal cycle reactions, a controller for controlling the fixed heat sources to temperatures corresponding to the thermal cycle reactions, and a mechanism for pressing against the container flat plate part on both sides thereof by means of blocks at least one side of which is the heat source, wherein the rotation mechanism and the mechanism for pressing the container flat part on both sides are controlled and the flat plate part is repeatedly brought sequentially into contact with the heat sources regulated at temperatures needed for the thermal cycles, thereby controlling the reaction solution in the aperture-for-reaction within the container flat place part at temperatures needed for the thermal reactions for durations needed for the thermal reactions, and performing nucleic acid amplification with the target thermal cycles.

According to embodiment 1, the nucleic acid amplification device is characterized in that the device sets and controls some of the fixed heat sources to a high temperature side from a heating target temperature when heating, or to a low temperature side from a cooling target temperature when cooling, by not less than a temperature difference defined by the equation below:

Y=5,000X³−900X²+50X+2.8 (a)

where Y=minimum temperature offset; X=Depth (mm) of the reaction solution×thickness (mm) of a container heat transfer surface. (The container heat transfer surface is defined as a film portion sandwiched between the surface of a container flat part in contact with a fixed heat source and the reaction solution).

According to other embodiment 1, the nucleic acid amplification device further comprises an optical measurement mechanism which measures optical changes in the aperture-for-reaction.

According to another embodiment 1, the nucleic acid amplification device is characterized in that in the blocks which press the described flat plate part on both sides thereof, the blocks are fixed heat sources and transparent members; the biochemical reaction is a nucleic acid amplification reaction, wherein the device has an optical measurement device consisting of a light emitting part that illuminates the reaction solution and a light receiving part that measures optical changes occurred, thereby permitting an optical measurement through the transparent member.

According to the 8th main aspect of the present invention there is provided a biochemical reaction kit including the container and a reaction solution containing an enzyme for a biochemical-reaction.

According to the 9th main aspect of the present invention, there is provided a reaction promoting device for providing a reaction solution held in a container with a predetermined thermal cycle thereby promoting a thermal cycle reaction such as PCR(polymerase chain reaction), LCR (ligase chain reaction), or the like, the device comprising: a thermal cycle heater which is disposed to face the reaction part of the container and which controls the temperatures of the reaction solution held in the container, within a predetermined thermal cycle time, to a high-temperature-side target temperature and a low-temperature-side target temperature thereby providing the thermal cycle; and a temperature controller part that controls the temperatures of the thermal cycle, in coordination with the predetermined cycle times, so as to offset the high temperature side thereof from the high-temperature-side target temperature by not less than a predetermined temperature difference, and offset the low temperature side thereof from low-temperature-side target temperature by not less than a predetermined temperature, wherein a control is made such that the temperature offset is not less than a temperature difference defined by the equation below:

Y=5,000X³−900X²+50X+2.8 (a)

where Y=minimum temperature offset ; X=Depth (mm) of the reaction solution×thickness (mm) of a container heat transfer surface. (The container heat transfer surface is defined as a film portion sandwiched between the surface of a container flat part in contact with a fixed heat source and the reaction solution.)

According to an embodiment 1, the reaction-promoting device is characterized in that the device comprises an apparatus that presses the container flat part on the front and back faces thereof, at least one side of the pressing apparatus structure being a fixed heat source, and at least one side the flat plate part's aperture-for-reaction serving as a 10 to 400 μm thick film-shaped heat transfer surface; and a mechanism to control the pressing apparatus, when allowing the fixed heat source to face the film heat source and bringing them into contact by the pressing apparatus, so that the contact of a film heat transfer surface, expanded due to a rise in the internal pressure of the aperture-for-reaction, occurs with the heat source in a stable manner.

According to another embodiment 1, the reaction-promoting device is characterized in that in order to introduce a reaction solution under pressure into a high-speed reaction part, the container further comprises a liquid inlet aperture having an open end which aperture is connected to an aperture-for-reaction via a liquid transport groove-like aperture in the container; and a mechanism that allows introducing the reaction solution from the open end of the liquid inlet aperture, transporting the reaction solution via the liquid transport groove-like aperture, then sealing the open end with a seal stopper, and pressing to permit a reaction to occur while the seal stopper is held pressed so as not to allow the seal stopper to come loose.

According to another embodiment 1, the reaction-promoting device is characterized in that at least one of the fixed heat sources has at least one heat source which is set at 90° C. or higher for enabling a hot start PCR and/or deactivation of reverse transcriptase.

According to yet another embodiment 1, the reaction-promoting device is characterized in that the device further has an optical detector device for reading out how the reaction has progressed by an optical change of the reaction solution.

According to another embodiment 1, the reaction-promoting device is characterized in that the device further comprises a power source for electrophoresis for subsequently separating nucleic acid amplification products by electrophoresis so as to judge how the reaction has progressed after nucleic acid amplification reaction has been performed.

According to yet another embodiment 1, the reaction-promoting device is characterized in that the device, in which a part holding a reaction solution in a flat part is flow channel-shaped, is for performing a flow type nucleic acid amplification reaction that runs an amplification reaction while the reaction solution is allowed to flow, further comprising a liquid transport controller for controlling the rate at which the reaction solution is transported.

According to the 10th main aspect of the present invention, there is provided a method of promoting a reaction that performs a nucleic acid amplification reaction with a thermal cycle such as PCR(polymerase chain reaction) or LCR (ligase chain reaction) wherein use is made of a container having a plurality of fixed heat sources set to specific temperatures corresponding to the predetermined temperatures of the heat cycle and having a flat plate part with an aperture-for-reaction for holding the reaction solution, with at least one side of the flat plate surface of the aperture-for-reaction being a heat transfer surface, and wherein in the reaction comprising the step of introducing the reaction solution into the aperture-for-reaction of the container and the step of repeatedly sequentially, as many times as the number of the cycles, bringing the aperture-for-reaction into contact with the fixed heat sources corresponding to the predetermined temperatures of the thermal cycles, there are heat sources in which the specific temperatures are offset toward a high-temperature side from a target temperature when heating, and toward a low-temperature side from a target temperature when cooling, by not less than a temperature difference expressed by the following equation:

Y=5,000X³−900X²+50X+2.8 (a)

where Y=a minimum temperature offset; X=Depth (mm) of the reaction solution×thickness (mm) of a container heat transfer surface. (The container heat transfer surface is defined as a film portion sandwiched between the surface of a container flat part in contact with a fixed heat source and the reaction solution.)

According to embodiment 1, the method is characterized in that the method uses a polymerase with a rate of 100 bases/sec or higher, such as TaKaRa Z-Taq™ and Toyobo KOD-Dash so as to assure a high speed reaction.

According to another embodiment 1, the method is characterized in that if the type of the targeted nucleic acid for amplification is RNA, the method comprises the step of bringing an RNA target into contact with fixed heat sources set at a reverse-transcription temperature for its transformation into an amplifiable DNA; and the step of subsequently bringing it successively into contact with fixed heat sources set for thermal cycles.

According to another embodiment 1, the method comprises the step of performing a plurality of optical measurements, as the thermal cycle progresses, for checking how the nucleic acid amplification progressed, thereby confirming the progress of the nucleic acid amplification.

According to another embodiment 1, the method comprises the step of identifying nucleic acid amplification products by reading out the optical changes of the reaction solution, while the temperature of the reaction solution is gradually raised after the nucleic acid amplification reaction so as to confirm that the nucleic acid amplification has correctly occurred.

According to yet another embodiment 1, the method comprises the step of performing electrophoresis after the nucleic acid amplification reaction.

According to yet another embodiment 1, the method is one wherein the nucleic acid amplification reaction is an emulsion PCR reaction.

According to yet another embodiment 1, the reaction container is such that the heat transfer surface is derived from a 10-400 μm thick film.

According to yet another embodiment 1, the reaction container is made of a light shielding material, or the interior wall of the aperture-for-reaction is shielded from light with a black or metal film for prevention of optical interference with the aperture-for-reaction so that the aperture-for-reaction does not does not optically interfere with neighboring apertures for reaction.

According to another embodiment 1, the reaction container is characterized in that a metal reflective film is mounted on either side of the flat plate thereof for enhancing the optical sensitivity by doubling the forward-and-backward path length of excitation light.

According to yet another embodiment 1, the reaction container is designed for the aperture part for reaction so that its reaction solution depth (mm)×container heat transfer surface thickness is 0.001-0.2.

According to yet another embodiment 1, the reaction container is characterized in that the reaction solution is filled dropwise into a concave part which has been formed by a method of forming a concave part in the flat place of the container or by drilling a through-hole in the container flat plate followed by sealing off one of the opening parts thereof with a film, thereafter by sealing the concave with 10-400 μm thick film, thereby forming a plurality of closed-system apertures for reaction independent from other apertures. It is preferred in this case for the reaction container to be built with at least one circumferential lip on an exterior wall face of the concave part and for that lip part to be hydrophobic.

Moreover, it is preferred in that case for the reaction container to have a center of rotation, and for the 20-200 closed-system apertures for reaction independent from other apertures to be circumferentially distributed entirely across the container flat part.

According to another embodiment 1, the reaction container is characterized in that the flat plate further comprises a liquid inlet aperture having an open end which aperture is connected to an aperture-for-reaction via a liquid transport groove-like aperture in the container; the reaction solution is introduced into the liquid inlet apertures and thereafter the reaction solution is transported to the aperture-for-reaction through the liquid transport groove-shaped aperture. In this case the aperture-for-reaction further has therefrom an air vent groove-like aperture being connected to an air vent port; and the container further has a seal aperture which is connected, in the container, to the liquid transport groove-like aperture and air vent groove-like aperture, to allow transporting the reaction solution from the liquid inlet aperture to the aperture-for-reaction via a liquid transport groove-like aperture and thereafter preferably transporting a seal liquid from the seal liquid aperture, thereby sealing off the liquid transport groove-like aperture and the air vent groove-like aperture. In addition it is preferred in this case for the seal liquid to be a high viscosity grease-like liquid including silicone grease, or a UV curable resin, or a thermosetting resin.

According to the 10th main aspect of the present invention, there is provided a kit which is used in the reaction promoting device and includes a container having a flat plate part holding a reaction solution containing a polymerase or ligase.

According to the 11th main aspect of the present invention, there is provided a reaction promoting device for providing, in a container having a reaction part holding a reaction solution, the reaction solution with a predetermined thermal cycle thereby promoting a thermal cycle reaction such as PCR(polymerase chain reaction), LCR (ligase chain reaction), or the like comprising: a thermal cycle heater which is disposed to face a reaction part of the container and provides the thermal cycle by controlling the temperatures of the reaction solution held in the container, within a predetermined thermal cycle time, to a high-temperature-side target temperature, a medium-temperature-target temperature, and a low-temperature-side target temperature; and a temperature controller part that controls the temperatures of the thermal cycle so as to offset the high-temperature-side thereof, from the high-temperature-side target temperature, by not less than a predetermined temperature difference, and so as to offset the low-temperature-side thereof, from the low-temperature-side target temperature, by not less than a predetermined temperature difference.

In addition, according to embodiment 1, there is provided a reaction-promoting device, wherein the device is so controlled that the temperature offset is not less than a temperature difference defined by the equation below:

Y=5,000X³−900X²+50X+2.8 (a)

where X=Depth (mm) of the reaction solution×thickness (mm) of a container heat transfer surface.

(The container heat transfer surface is defined as a film portion sandwiched between the surface of a container flat part in contact with a fixed heat source and the reaction solution).

According to another embodiment 1, the thermal cycle heater comprises a high temperature part, a medium temperature part, and low temperature part; and a drive part which provides a predetermined thermal cycle to the reaction solution held in the reaction part by displacing the relative position of the thermal cycle heater with respect to the container, wherein the drive part controls the period of time during which the container faces the heater by having it coordinated with the predetermined thermal cycle time.

According to another further embodiment 1, the high temperature part, medium temperature part, and low temperature part are disposed in a circumferential direction; the drive part drives the container or the heater so that the relative position of the container with respect to the heater is displaced in a rotational circumferential direction.

According to another embodiment 1, the drive part intermittently causes a relative displacement of the thermal cycle heater and the reaction container while they are in contact with each other, thereby causing the reaction solution held in the container to reside for a predetermined time in the thermal heater's high temperature part, medium temperature part, and low temperature part, respectively.

According to another embodiment 1, the control part adjusts the speed with which a specific part on the container moves until it faces the low temperature part of the thermal cycle heater, the speed with which it moves from the low temperature part to the medium temperature part, and the speed with which it exits from the medium temperature part, thereby holding the thermal history applied to the specific part constant at least in regard to the low and medium temperatures.

According to another embodiment 1, the drive part continuously causes a relative displacement of the thermal cycle heater and the reaction container while they are in contact with each other.

According to another embodiment 1, the high temperature part, medium temperature part, and low temperature part are disposed along a linear direction, and the drive part drives the container or the heater so that the relative position of the container with respect to the heater is displaced in a linear direction.

According to another embodiment 1, the control part drives, when displacing the thermal cycle heater from the reaction container, so as to separate the cycle heater from the reaction container and displace them while separated, and thereafter to bring the cycle heater into contact with the reaction container.

According to yet another embodiment 1, the device further comprises an optical detector apparatus for reading out how well the reaction has progressed; and the container has at least part thereof formed of a light shielding material or is tinted to shield light for preventing the optical interference of a plurality of the reaction solutions.

According to yet another embodiment 1, the container uses an optically transparent pressing member as a member to press the container; and an optical measurement is made through the transparent pressing member.

According to another embodiment 1, there is provided a reaction promoting device wherein the device has an apparatus that presses the container flat plate on its front and back faces; at least one side of the pressing apparatus structure is a fixed heat source; at least one side the reaction part of the flat part is a 10-400 μm thick film heat transfer surface; and the fixed heat source is allowed to face the film heat transfer surface and is brought into contact therewith by the pressing apparatus, thereby heating; and a rise in the internal pressure of the reaction part causes the film heat transfer surface to expand, thereby securing a stable contact of the container with the heat source.

According to yet another embodiment 1, the container is a reaction container that has a flat plate part wherein a concave part is formed into the flat plate part thereof; a reaction solution, which is an aqueous solution, is placed in the concave part; thereafter the concave part is sealed, thereby sealing off the reaction solution; and a fixed heat source set at a temperature corresponding to the temperature needed for the reaction is brought into contact with the flat plate part thereby performing nucleic acid amplification reaction, a thermal reaction; and wherein at least one circumferential lip is formed on an upper end face of the concave part; and both the circumferential lip part and the lower face of the seal member are made hydrophobic, thereby allowing the nucleic acid amplification reaction to be stably performed.

According to yet another embodiment 1, the container further comprises a liquid inlet aperture with an open end for introducing a reaction solution under pressure into a reaction part, which aperture is connected to the reaction part via a liquid transport groove-shaped aperture in the container; and a mechanism that allows introducing the reaction solution through the open end of the liquid inlet aperture, then transporting the reaction solution to the aperture-for-reaction through the liquid transport groove-shaped aperture, thereafter sealing the open end with a seal stopper, and pressing to permit a reaction to occur while the seal stopper is held under pressure so as not to cause the seal stopper to come loose during the reaction.

According to yet another embodiment 1, the seal stopper contains an air groove extending from the middle part of the seal stopper toward the lower part thereof.

According to yet another embodiment 1, the container contains an air vent groove-shaped aperture connected to the reaction part; a seal liquid aperture filled with a seal liquid for allowing the seal liquid to flow; and a groove-shaped aperture that connects the seal liquid aperture to the air vent groove-shaped aperture and the liquid inlet aperture, and wherein after transport of the reaction solution from the liquid inlet aperture to an aperture-for-reaction, the seal liquid is transported from the aperture filled with the seal liquid, thereby introducing the seal liquid into both the groove-shaped aperture on the liquid inlet aperture side of the aperture-for-reaction and the groove-shaped aperture on the air vent side groove-shaped aperture.

According to yet another embodiment 1, the seal liquid is composed of a high viscosity grease-like liquid including silicone grease, or a UV curable resin, or a thermoset resin.

According to yet another embodiment 1, the device, in which a part holding a reaction solution in a flat part is flow channel-shaped, is for performing a flow type nucleic acid amplification reaction that runs amplification reaction while the reaction solution is allowed to flow, further comprising a liquid transport controller for controlling the rate at which the reaction solution is transported.

### BRIEF DESCRIPTION OF THE DRAWINGS

FIG. 1 is a drawing showing the types of nucleic acid amplification methods.

FIG. 2 is a drawing for explaining a typical existing high-speed device.

FIG. 3 is a drawing for explaining the PCR time for each heat source of a conventional device.

FIG. 4 is a schematic diagram showing the device of a first embodiment of the present invention.

FIG. 5 is a schematic diagram showing the device of a first embodiment of the present invention.

FIG. 6 is a schematic diagram showing a reaction container of a first embodiment of the present invention.

FIG. 7 is a schematic diagram showing the arrangement of heat sources in a first embodiment of the present invention.

FIG. 8A and FIG. 8B are schematic diagrams showing a reaction vessel of a first embodiment of the present invention.

FIG. 9A to FIG. 9C are schematic diagrams showing the process for installing the reaction container of a first embodiment of the present invention.

FIG. 10 is a schematic diagram showing an enlarged view of a reaction vessel of a first embodiment of the present invention.

FIG. 11 is a drawing for explaining the theory of a first embodiment of the present invention.

FIG. 12 is a drawing for explaining the thermal offset method of a first embodiment of the present invention.

FIG. 13 is a schematic diagram showing a fluorescent light measurement device of a first embodiment of the present invention.

FIG. 14 is a schematic diagram showing the arrangement of the fluorescent measurement device of a first embodiment of the present invention.

FIG. 15 is a block diagram for explaining the control of a first embodiment of the present invention.

FIG. 16 is a flowchart showing the operation of a first embodiment of the present invention.

FIG. 17 is a flowchart showing the operation of a first embodiment of the present invention.

FIG. 18 is a flowchart showing the operation of a first embodiment of the present invention.

FIG. 19 is a flowchart showing the operation of a first embodiment of the present invention.

FIG. 20 is a top view showing a reaction container of another embodiment of the present invention.

FIG. 21A and FIG. 21B are schematic diagrams showing a reaction vessel of another embodiment.

FIG. 22 is a top view showing a reaction container of another embodiment of the present invention.

FIG. 23A and FIG. 23B are drawings showing a reaction vessel of another embodiment of the present invention.

FIG. 24 is a top view showing a reaction container of another embodiment of the present invention.

FIG. 25 is a top view showing a reaction container of another embodiment of the present invention.

FIG. 26 is a drawing showing the arrangement of heat sources in another embodiment of the present invention.

FIG. 27 is a cross-sectional drawing showing an enlarged view of a reaction vessel of another embodiment of the present invention.

FIG. 28A and FIG. 28B are a top view and side view, respectively, showing a device of a second embodiment of the present invention.

FIG. 29 is a front view showing a device of a second embodiment of the present invention.

FIG. 30 is a drawing showing a reaction container that is used in a second embodiment of the present invention.

FIG. 31 is a side view showing a device of a third embodiment of the present invention.

FIG. 32A to FIG. 32C are drawings showing a reaction container that is used is a third embodiment of the present invention.

FIG. 33 is a flowchart showing the operation of a third embodiment of the present invention.

FIG. 34 is a flowchart showing the operation of a third embodiment of the present invention.

FIG. 35 is a flowchart showing the operation of a third embodiment of the present invention.

FIG. 36 is a side view showing a device of a fourth embodiment of the present invention.

FIG. 37 is a top view showing a device of a fourth embodiment of the present invention.

FIG. 38 is a front view showing a device of a fourth embodiment of the present invention.

FIG. 39 is a flowchart showing the operation of a fourth embodiment of the present invention.

FIG. 40 is a drawing showing the temperature differences that occur in the heat sources.

FIG. 41 is a drawing explaining the temperature setting of each temperature heat source, and the temperature that each reaction vessel receives by that construction.

FIG. 42 is a drawing explaining improved temperature settings for each temperature heat source, and the temperature that each reaction vessel receives by that construction.

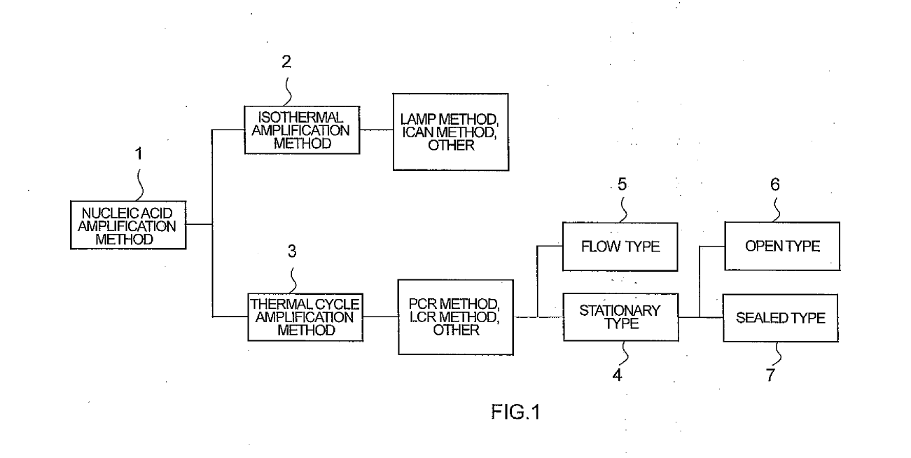

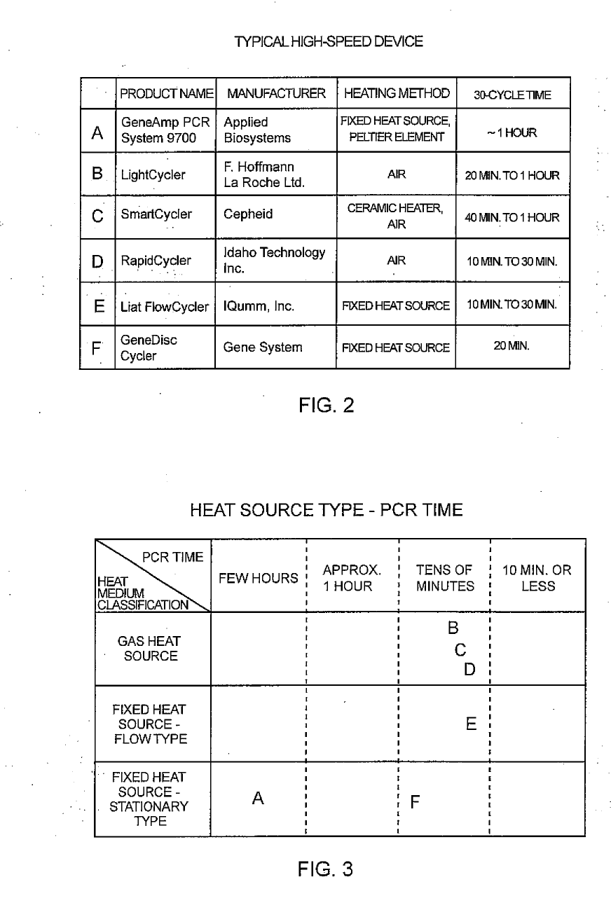

### DETAILED DESCRIPTION OF THE PREFERRED EMBODIMENTS

Embodiments of the present invention are hereafter explained with reference to the drawings.

As described above, performing a high-speed nucleic acid amplification in the 5 minutes or less called for in the market requires making overall improvements of all the elements about PCR, such as the heat source type, container shape, device control, enzymes to be used and the like.

(Basic Technical Matter Improved by the Present Invention)

Technical requirements for a high speed and stable execution using PCR first as an example are enumerated below:
a. Shorten appreciably the time needed for changing the temperature up and down . . . Speedup.
b. Shorten appreciably the delay in temperature changes between the heat source and the reaction solution . . . Speedup
b-1. Secure the contact between the heat source and reaction container . . . Stabilization
b-2. Reduce the container surface layer thickness for securing heat transfer from the container surface to the reaction solution and Reduce the liquid thickness for securing heat conduction within the liquid. . . . Speedup
b-3.Control so as to accelerate heat transfer within the reaction solution . . . Speedup

(Adoption of a Fixed Heat Source-Stationary Type)

In regard to Item a, with the present invention it was decided to adopt a fixed heat source-stationary type, to prepare a plurality of fixed heat sources with different set temperatures corresponding to each temperature and to provide the reaction solution with thermal cycles by moving the reaction container relative thereto. This thereby greatly minimizes the times for heating and cooling the heat source so that the only the time needed for heat transfer to the reaction solution in the reaction container is that needed for changing the reaction solution temperature up and down.

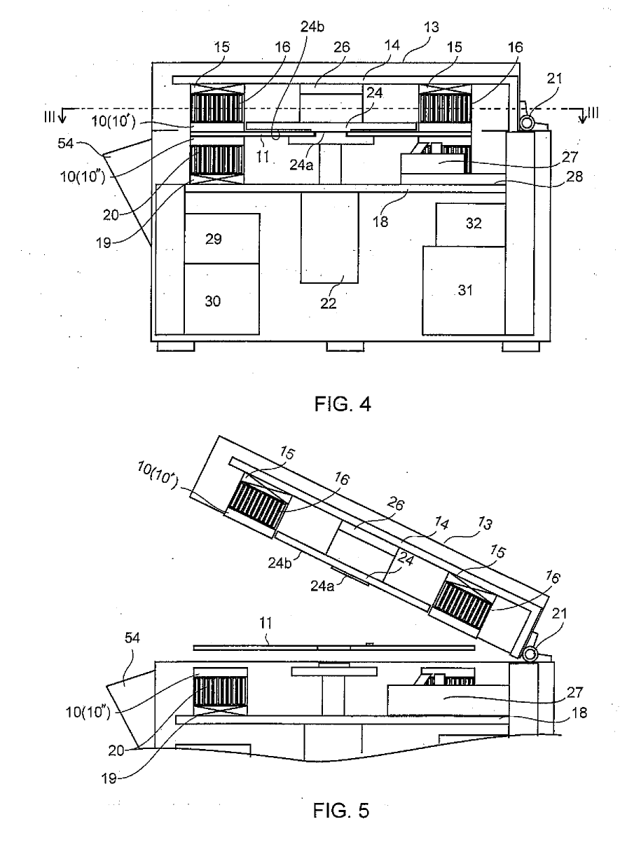

FIG. 4 is a side view illustrating the entire outline of a reaction-promoting device of the 1st embodiment of the present invention.

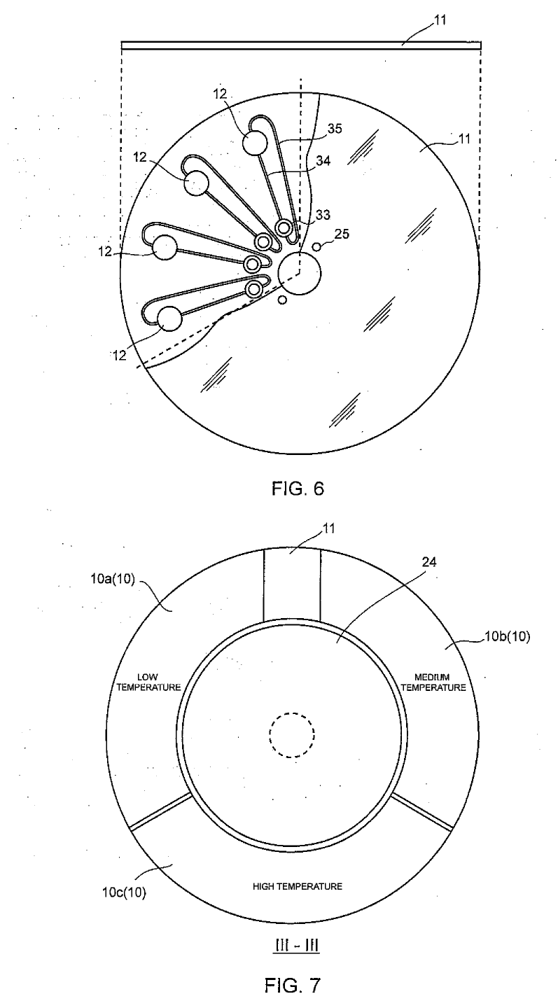

In FIG. 4, Reference symbol 10 shows a fixed heat source and 11a reaction container. As shown in FIG. 6, the reaction container 11, when viewed from above, is seen to be disk-shaped and the part marked with 12 is a reaction chamber to be filed with a reaction solution. In addition, as shown in FIG. 7 the fixed heat source comprises three different blocks, 10a to 10c, which are set at low temperature, medium temperature, and high temperature, as formed in sectors to accommodate the shape of the reaction container 11, and is so configured that rotation of the reaction container 11 against the heat source provides the reaction solution in the reaction chamber 12 with thermal cycles.

Returning to FIG. 4, the fixed heat source 10 is disposed so as to sandwich the reaction container 11 between an upper heat source 10′ and lower heat source 10.″ The upper heat source 10′ is held against a top frame 14 built on an upper lid 13 via a spring 15 and a heat dissipation block 16; and the lower heat source 10″ is similarly held through the spring 19 and the heat dissipation block 20 against frame 18 of main body side 17 via a spring 19 and a heat dissipation block 20. For the heat source 10, a Peltier element is adopted for the ease and accuracy of control.

The upper lid 13 and the main body 17 are connected with a hinge member 21 and as shown in FIG. 5, the hinge member 21 allows the upper lid 13 to be openable and closable with respect to the main body 17 so that opening the upper lid 13 can release the reaction container 11. On the other hand, as shown in FIG. 4, against the main body 18 is held a main motor 22 for driving the rotation of the reaction container 11, with an axis-of-rotation 22a thereof projecting upwardly; on the top end of the axis-of-rotation 22a is provided a holding table 23 for holding the reaction container 11. It is so arranged that the reaction container is installed as positioned on a table 23, where closing the upper lid 13 allows the upper and lower heat dissipating blocks 10′ and 10″ to sandwich the peripheral edge part of the reactor 11.

The central part of the reaction container 11 is configured to be clamped by the holding table 23 and the upper side holding block 24 built to the upper lid frame 14. The upper side holding block 24 has a first face 24a for pressing against the central part of the container and a second face 24b to be described later, built higher than the first, for pressing the seal stopper. The holding block 24 is configured so as to be held against the upper side frame 14 via a spring 26 thereby pushing resiliently the reaction container 11 against the holding table 23.

Further, this device is equipped with a fluorimeter 27. The fluorimeter is for detecting the reaction that has occurred in the reaction solution by illuminating the reaction solution in the reaction container 11 with excitation light and measuring the reflected fluorescence. The fluorimeter 27 is held on a movable table 28 and can be driven for positioning in a radial direction of said reaction container.

Moreover, this device is equipped with a heater temperature controller part 29, a rotary motor controller part 30, a fluorimeter controller part 31, and a power supply 32 in said main body 17.

A detailed configuration of this device is explained along with its operation below:

(Reaction Container)

With reference to FIGS. 6 and 8 to 10, the reaction container 11 is explained in detail first.

As shown in FIG. 6, the reaction container 11 is provided with four reaction chambers 12 which are built over a 120 degree range in a circumferential direction about 25 degrees apart from each other. A liquid transport hole 33 is provided, for transporting the reaction solution to each of the reaction chambers 12, at a central part side of the reaction container, corresponding to each reaction chamber 12. The reaction chamber 12 and liquid transport hole 33 are connected by a liquid transport path 34 and air release path 35.

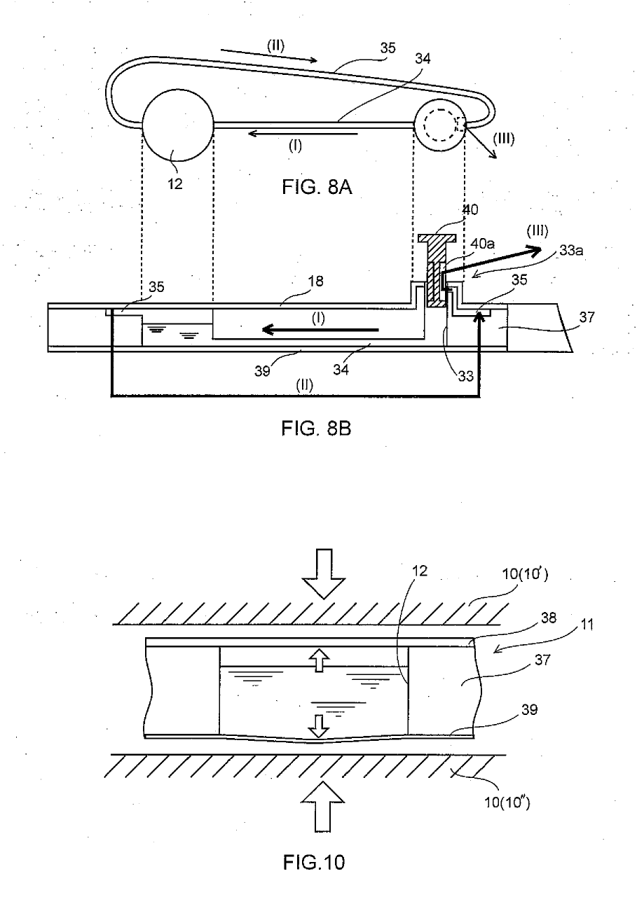

FIGS. 8A and 8B show one of said reaction chambers 12, along with the cross section of the part corresponding thereto. The reaction container 11 is constituted of a main body base plate 37 made of polycarbonate, a blocking plate 38 laminated to the top face of the base plate 37, likewise made of polycarbonate, and a 200 μm thick stretchable film adhered to the lower face thereof. Said reaction chamber 12 and liquid transport hole 33 are built to penetrate through said main body base plate 37; said liquid transport path 34 is built as said 100 μm thick groove on the back side of the main body base plate 37 and the air release path 35 is built as a 100 μm deep groove on the upper side thereof, respectively. In addition, covering said upper and lower faces with the blocking plate 38 and stretchable film 39, allows the reaction chamber 12 and the liquid transport hole 33 to be blocked off, respectively. Separately, the upper face of the portion which constitutes the above-mentioned liquid transport 33 is configured as an upward projection part 33a such that said air release path 35 communicates with that projected portion 33a. The reason for such a configuration is to vent the air in said reaction chamber 12 via the air release path 35, while vacating the air within the reaction container 11 with a seal stopper marked with 40 in the Figure as the reaction solution is being transported from the liquid transport hole 33 to the reaction chamber 12 via the liquid transport path 34.

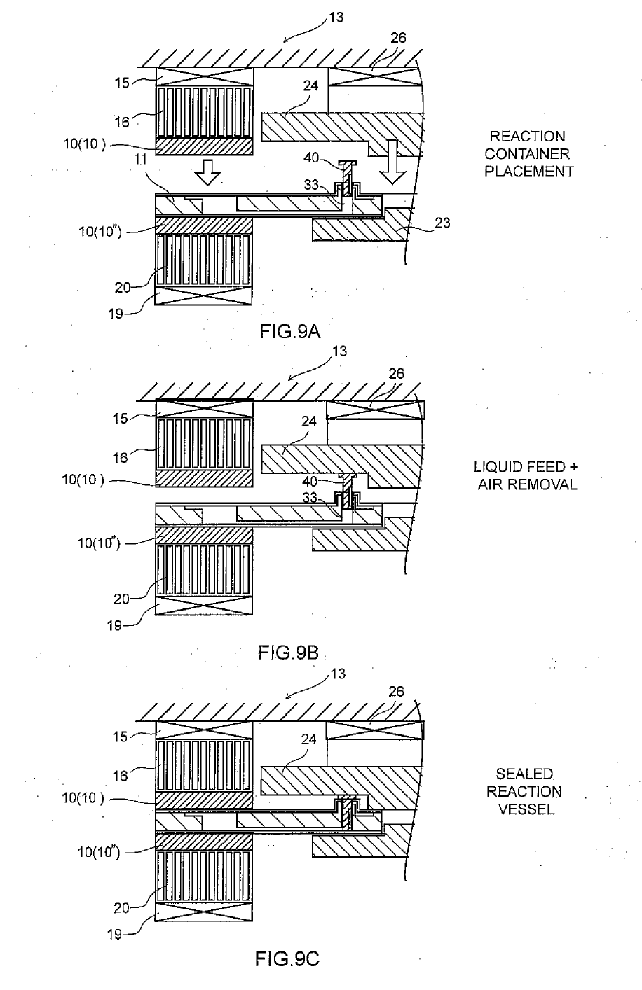

Next, the flow of steps, the transport of the liquid, air venting, and closure, is explained with reference to FIGS. 9A-C.

First, as shown in FIG. 9A, the reaction container 11 with the reaction solution filled in the liquid transport hole 33 is placed on the holding table 23 at the main body. At this time, the seal stopper 40 is provided with its tip being inserted into the liquid transport hole 33 and with the atmospheric-communication hole of the container 11 being released to the atmosphere by an air vent path of the seal stopper 40.

Then, the upper lid is released as in FIG. 5 to allow placing the reaction container at the main body side.

Next, on shutting the upper lid 13, as shown in FIG. 9B, the seal stopper 40 is press-fitted into the liquid transport hole 33 of the reaction container 11 by the upper side holding block 24. FIG. 8B shows this as enlarged. The reaction solution charged in the liquid transport hole 33 moves to the reaction chamber 12 through liquid transport path 34, and the reaction solution is filled in the reaction chamber 12 (arrow (I)). At this time, through the air release path 35 formed between the upper blocking plate 38 and the base plate 37, the air in the reaction aperture is forced into the above-mentioned liquid transport hole 33 (arrow (II)) and is vented to the atmosphere through the air vent path 40a of the seal stopper 40 (arrow (III)).

As shown in FIG. 9C, upon completely shutting the upper lid 13, due to the resiliency of spring 26 mounted in the holding block 24, the seal stopper 40 is further lowered; the 40a of the seal stopper 40 is also immersed into liquid transport hole 33, whereby air will no longer escape thereafter. Further, with the seal stopper 40 being completely pushed in, the interiors of liquid transport path 34 and the reaction chamber 12 are placed under an optimum pressure, i.e., a pressurized state above atmospheric pressure.

Moreover, since the seal stopper 40 is pressed down by a second face 24b of the holding block 24 and the upper surface of the reaction container 11 is pressed by a first face 24a at this time; the reaction container 11 including the seal stopper 40 is fully gripped. This enables the reaction container 11 to be driven to rotate, and at the same time prevents the seal stopper 40 from coming loose during the rotation thereof.

The heating efficiency of the reaction solution in the reaction chamber 12 is improved in the present invention by having the reaction chamber 12 filled in with the reaction solution and having inside of the reactor chamber 12 pressurized. FIG. 10 illustrates this principle.

The first novel feature of the reaction container 11 is that the reaction chamber 12 part is clamped between the upper and lower heat sources 10′ and 10″ thereby enhancing the heat transfer efficiency. In particular both the upper and lower faces or either of them that block the reaction chamber 12 is thin-walled, with the thin wall member serving as a respective heat transfer surface. The upper and lower fixed heat sources 10′ and 10″ are configured to be mounted in this embodiment, thereby pressing the reaction container 11, but the fixed heat source may be either on both sides or on one side so long as the heat source is such that the reaction solution faces, and is in contact with, the heat transfer surface. This example sets the heat transfer surface on the lower face side in which the reaction solution accumulates, where the member constituting the heat transfer surface is required to be thin. If it is too thin, there is the risk of breakage dictating the need for a minimum thickness of 10 μm. A thickness greater than 400 μm will reduce the heat transfer efficiency, which is not favorable for speedup. This sets the heat transfer surface thickness to be 10 μm to 400 μm, preferably 50 μm to 300 μm, most preferably about 150 μm. Moreover, in consideration of heat transfer to the reaction solution, the liquid depth of the reaction solution in the reaction chamber 12 is preferably not more than 1 mm, optimally about 500 μm so as not to increase the heat capacity of the liquid per the heat transfer surface.

Moreover, in order to enhance the heat transfer efficiency the heat transfer surface is constituted of a stretchable film 39 and the reaction chamber 12 is held above atmospheric pressure during the processing so as to cause the film 39 to expand toward the heat source 10″, thereby increasing contact with the heat source 10. This aspect is further detailed below.

(Securing Contact Stability)

That is, in order to speed up a PCR, it is critical to maintain reliable contact of the fixed heat source 10 with the reaction chamber 12 portion of container 11 during motion through as many as 30 cycles. A reaction by the fixed heat source 10 and 11 such as the present technique makes the stability of that contact an important factor.

Usually this calls for pressing the container 11 into contact with the fixed heat source 10. However the container 11 and the fixed heat source 10, by virtue of both being solid, will end up generating minute voids due to slight interfering unevenness on their surfaces. The low thermal conductivity of air present in these voids greatly reduces stability in heat transfer. In order for many reaction chambers to experience identical heat histories over 30 or more cycles, it is nearly impossible to bring the two into perfect contact by a conventional planar pressure contact alone. The securing of this contact is a decisively important technique for a biochemical reaction using a fixed heat source and solid container such as the present process.

Furthermore, a diagnostic application, for securing the assurance of the results, often runs tests along with a negative control (to detect nothing if a test target is absent) and a positive control (to know that the test proceeded well with no problems even if a test target is absent). That is, a test for one item for a single specimen ends up being three tests for one item. There also is much need for simultaneously testing a large number of specimens. When the flat plate part of a device has a large number of apertures for reaction arranged, the heat transfer surfaces of these reaction parts themselves generate some unevenness, which interactively produces in practice reaction parts in and out of contact therewith.

On the other hand, the present invention raises the internal pressure of a reaction chamber 12 using a blocking member made of a stretchable film 30 and allows the film 39 to expand in an exterior direction, thereby enabling the films 39 of many reaction chambers to be brought stably into contact with the solid.

It was learned from the foregoing that in order for many reaction chambers 12 formed in the container 11 to achieve a uniform and stable contact in all the reaction chambers 12 with the fixed heat sources 10 and perform nucleic acid amplification reaction in a stable manner, this is accomplished by using an aperture-for-reaction having a thin heat transfer surface or a soft stretchable film blocking member (stretchable film 39 in this embodiment) and by achieving conditions under which the internal pressure of the reaction chamber 12 is above atmospheric pressure, whereby the stability of the reaction can be secured.

That is, since the container base plate 11 itself is hard and has insufficient flatness, there is a beneficial effect in that the container blocking members (flexible film 39) of the many reaction chambers 12 present therein expand, thereby allowing the mutual contact force to automatically line up all the reaction chambers, by deformation of the blocking film 39, to come in contact with the heat source 10.

Although in this embodiment, the base plate 37 of a container 11 has a stretchable film 39 of a different material adhered thereto, there is no limitation to it. The blocking member therefor may be the same material as that of base plate 37,which is made stretchable by reducing its thickness. In addition, a stretchable film can be chosen from an olefin-based resin, polyester based resin, polyurethane resin, silicone based resin, or a fluororesin and the like, but from among these, fluororesin is most preferred in view of softness and strength.

There are various methods for producing the reaction container 11 having a stretchable film 39 as below. For example, there is a method as in this embodiment of adhering a stretchable film 39 to a container base plate 11 having reaction chambers 12. There is also a method of fabricating a container base plate 37 itself from a material which may be expected to be stretchable when thin, thereby forming the stretchable film 39 by single-piece molding.

Moreover, this embodiment secures contact of the fixed heat source with the stretchable film by keeping the pressure inside the reaction chamber 12 above atmospheric pressure, but the method of keeping the pressure inside the reaction chamber 12 high is not limited to that of this embodiment. That is, this embodiment achieved this using a seal stopper 40 and liquid transport path 34, without being limited thereto. This effect can be achieved with a container 11 having no liquid transport path but having independent reaction chambers by introducing the reaction solution at ordinary temperature and pressure followed by simply sealing off the individual reaction chambers with a surface blocking member. This is because the nucleic acid amplification reaction is all run above ambient temperature, in an aperture-for-reaction where the liquid and/or gas in the reaction aperture, as heated, expands, thereby securing contact with the fixed heat source.

The present inventor also carried out the following method in order to ensure this. That is, when a container 11 it is fed with a reaction solution, the lower face of the container is depressurized thereby causing the blocking film 39 at the lower face thereof to project downward, followed by feeding the reaction solution, adhering a blocking member at the upper face side, and stopping the depressurization. The resultant reaction container 11 will be such that the internal pressure of the reaction chamber 12 exceeds atmospheric pressure before being subjected to the reaction, thereby further enabling more reliable stabilization of the reaction in the thermal reaction.

(Novel Features for Heat Source Temperature Control)

This embodiment also includes an improvement for the control method of the heat source temperature, in addition to the improvements over the shape of the above reaction container 11 and the like; before an explanation of how this device works, its principle is provided here.

Previous patents and references have many examples describing the thicknesses of container bottoms and the depths of liquid reaction mixtures. However, there is a limit to the speeding up of the thermal cycle by merely relying on the thickness and depth alone. This has prompted these inventors to go back to the aperture-for-reaction basis of heat transfer and study the speedup by paying attention to the temperature difference between the heat source and members being heated.

In other words, all of the conventional technology was such that heating was performed by setting the temperature setting of the heat source at or near the target heating temperature for the liquid reaction mixture, however, in the present invention, by setting the temperature of the heat source such that it is offset a specified temperature on the high temperature side from the target temperature when raising the temperature, and setting it such that it is offset a specified temperature on the low temperature side from the target temperature during cooling, it was found that the time for applying a thermal cycle to the liquid reaction mixture could be greatly shortened.

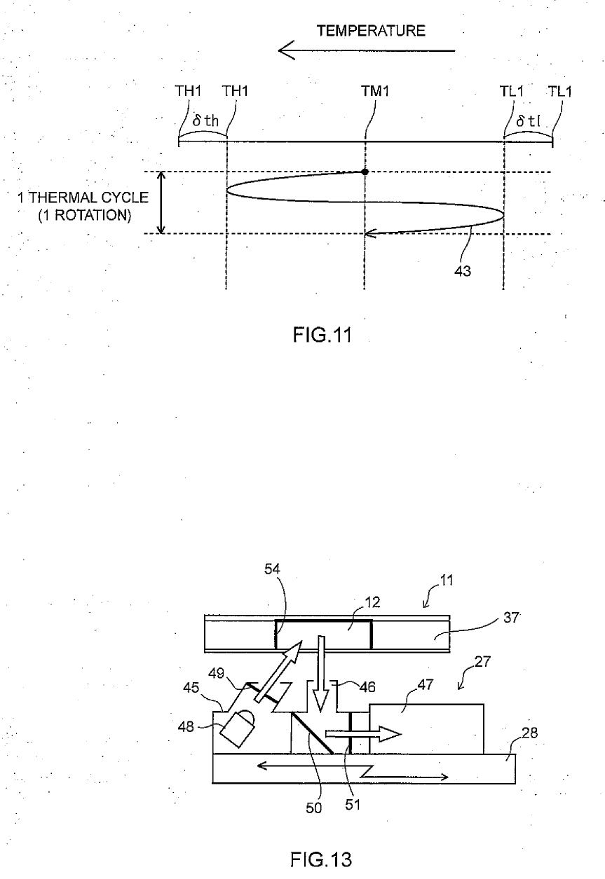

This is illustrated and shown in FIG. 11.

That is, as the thermal cycle time becomes shorter, unless the difference in the heat energy of the heat source 10a on the low-temperature side and the heat source 10c on the high-temperature side is increased, it is not possible to raise or lower the temperature at high speed. In FIG. 11, the temperature setting TH1 of the heat source 10c on the high-temperature side is increased with respect to the target temperature TH0 for the liquid reaction mixture on the high-temperature side by just an offset temperature amount δth, and the temperature setting TL1 of the heat source 10a on the low-temperature side is decreased with respect to the target temperature TL0 for the liquid reaction mixture on the low-temperature side by just an offset temperature amount δtl. In addition, by rotating the reaction container 11 in connection with the thermal cycle time, the temperature transition as shown by 43 in FIG. 11 is realized.

How to set these offset temperatures 8th and δtl, or in other words, how to set these offset temperatures so that nucleic acid amplification can be performed in 5 minutes or less as desired in the clinical field was analyzed by numerical calculation of the heat transfer.

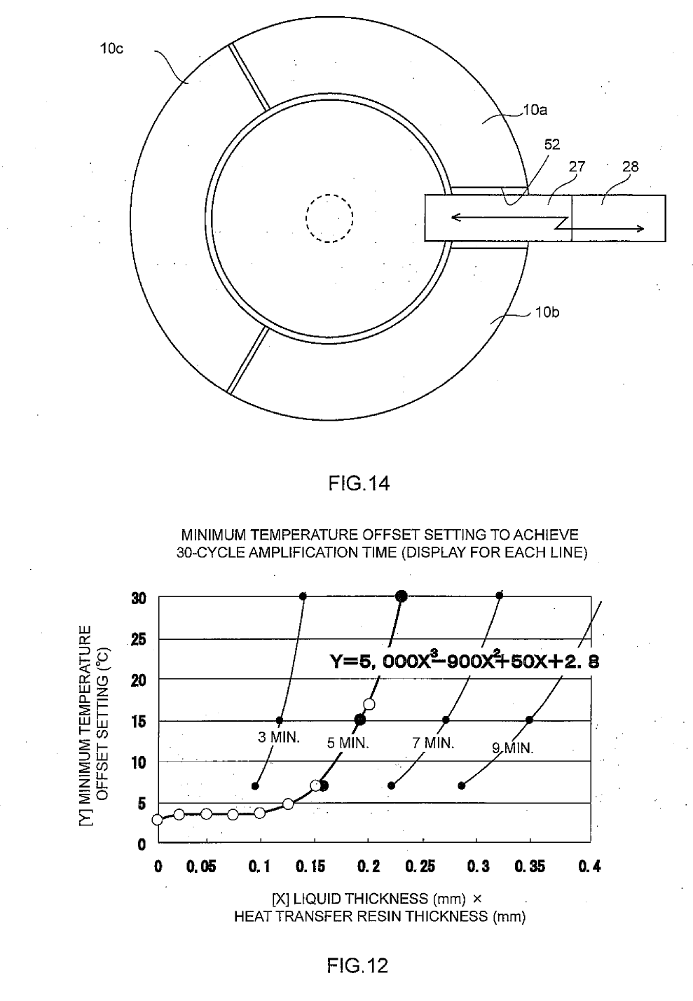

A relationship between the (thickness of the container's heat transfer surface x depth of the liquid reaction mixture) and the minimum required excess temperature setting that was obtained as a result is shown in FIG. 12. From the obtained result, an approximate expression was created for the result of 30 cycles in 5 minutes, with the following result being obtained.

Y=5,000X³−900X²+50X+2.8 (a)

where, Y=minimum excessive set temperature, X=depth of the liquid reaction mixture (mm)×thickness of the container's heat transfer surface (mm).

The heat transfer surface of the container is the film-like portion of the flat plate section of the container that is located between the front face that comes in contact with the fixed heat source and the liquid reaction mixture.

The expression can be found as described below.

[Assumption]

PCR liquid reaction mixture having a thickness L1 cm is located on a Ti° C. heat source with a resin film having a thickness Lp cm per unit area of 1 cm² at a temperature To° C. located between them. The amount of heat Q that is transferred through each unit cm² is typically given by the following equation.

Q=λ/Lp*(Ti−To)

where Q: amount of heat that is transferred (cal/sec)
λ: heat transfer rate (cal/cm•sec•° C.)
Lp: thickness of the resin film (cm).

The heat transfer rate of the liquid reaction mixture (water, liquid) is several times the heat transfer rate of PC or ABS resin, and with the liquid having liquidity and being in a thin film-like state, it is assumed that the heat that passes through the resin wall is used as is to raise the temperature of the liquid.

δQ1λ/Lp*(Ti−To)*δt

With the specific heat of water taken to be 1, the rise in temperature is given to be dT1=dQ1/L1.

δQ2λ/Lp*(Ti−To+dT1)δt

To perform numerical calculation of the heat transfer:

δQn=λ/Lp×(Ti−(To−ΣdT1˜n))×ιt (b)

δTn=δQn/L1 (c).

Adding the idling time of the machine together with the result of numerical calculation using equations (b) and (c), a graph of the required excess set temperature is obtained from the relationship with the time required for 30 samples (depth of the liquid reaction mixture× thickness of the container's heat transfer surface) and shown in FIG. 12. (For λ, 0.001 cal/cm•sec•° C. of silicone resin having a relatively high heat transfer rate is used even in resin.) In FIG. 4, the calculation results at 5 minutes are shown by black dots, and the calculation results from the approximation equation are shown by white dots.

From the above, as control for accelerating the heat transfer of item b-3, by taking the product of the depth of the liquid shown in millimeters and the thickness of the blocking member to be X, and substituting that X into equation (a) according to the thermal cycle time, a high-speed reaction is possible that offsets the temperature of the heat source by at least that obtained temperature or greater. Here, the value of X is not limited, however, a value of 0.001 to 0.2 is preferred. A value of 0.01 to 0.15 is even more preferable.

A detailed example of the setting will be described. In normal PCR, a high temperature of 95° C., low temperature of 55° C. and medium temperature of 75° C. are necessary. In this case, this is greater than the value of equation (a), and the offset temperature is set to about 3 to 30° C. according to the thermal cycle time. Here, in an example where the differences in the offset temperature settings δth, δtl are 15° C., TH1 is set to 110° C., TL1 is set to 40° C. and TM1 is set to 75° C., and the cycle of bringing each in contact with the respective fixed heat source 10a to 10c is repeated 30 to 40 times. The time required for moving toward each heat source 10a to 10c is approximately 0.6 seconds, so in the case of 300 bp, the time stopped above each heat source is TH: 0 seconds; TL: 0.5 seconds and TM: 3 seconds. Thus amplification is completed in about two and a half minutes. In this way, amplification can be accomplished with sufficient speed even in the clinical emergency field.

(Movement Speed to Each Temperature Heat Source)

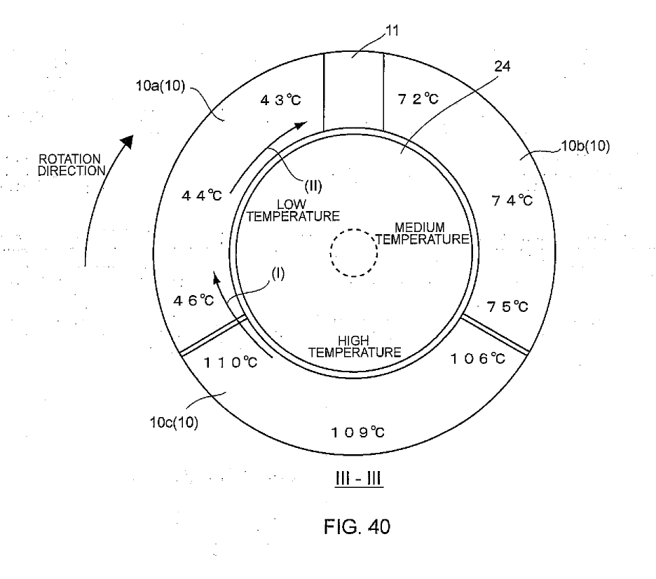

The inventors also performed further innovation related to the speed of moving to each of the temperature heat sources. FIG. 40 is a drawing showing that such innovation is necessary.

In this figure as well, the fixed heat source on the low-temperature side 10a to the fixed heat source on the high-temperature side 10c are arranged around the circumferential direction as described above, and the aforementioned circular reaction container 11 rotates so as to come in contact with these fixed heat sources 10a to 10c. With this construction, as the container 11 moves from the fixed heat source 10c on the high-temperature side to the fixed heat source 10a on the low-temperature side, from the fixed heat source 10a on the low-temperature side to the fixed heat source 10b on the middle-temperature side, and from the fixed heat source 10b on the middle-temperature side to the fixed heat source 10c on the high-temperature side by rotating that reaction container 11, the heat that the container 11 obtained before moving has an effect on the fixed heat source 10a to 10c to which it moves, and also has an effect on the uniformity of the temperature of the container 11.

More specifically, for example, when the portion of the container 11 that was heated by the fixed heat source 10c on the high-temperature side moves toward the fixed heat source 10a on the low-temperature side as shown by arrow I, the heat from the container 11 is quickly absorbed by the fixed heat source 10a on the low-temperature side, causing the temperature of the fixed heat source 10a on the low-temperature side to drop. Moreover, as shown by the arrow II, when a portion of the container 11 reaches the front end of the fixed heat source 10a on the low-temperature side, the temperature of the container 11 that was initially heated to a high temperature drops to below that temperature. As a result, the rise in temperature of the front end of the fixed heat source 10a on the low-temperature side caused by the heat of the container 11 becomes less than the rise in temperature of the portion in back end of the fixed heat source 10a on the low-temperature side, and as shown in FIG. 40, a temperature difference occurs between the front end (43° C.) and the back portion (46° C.) of the fixed heat source 10a on the low-temperature side, and with that, a difference in cooling speeds of the reaction wells of the container occurs.

In other words, the temperature of the heat source itself is controlled, so this temperature difference disappears over time, however, in an ultra-high-speed PCR device that takes advantage of dynamic heat movement, there is a possibility that, due to this small temperature difference in the heat source temperature, a difference will also occur in the reaction results of a plurality of wells. This phenomenon is a unique problem of contact rotation type ultra-high-speed PCR, and is a serious problem that should be avoided in order to maintain stability of the PCR reaction in each reaction well.

With regards to this temperature difference in the heat source, in a normal thermal cycle PCR device, the temperature of each heat source is set to a target temperature, and by having contact over a long period of time with a heat source at a set temperature, each well becomes the same temperature even though there is small time difference, so there is no problem.

However, in the ultra-high-speed PCR of this embodiment, in order to raise and lower the temperature quickly, the temperature settings of the heat sources are set at temperatures (offset temperatures) such that heating or cooling is performed beyond the target temperatures, and since it is a method of using dynamic heat movement in which the time that the container is stopped above a heat source is extended, the temperatures of the reaction wells exceed the target temperatures, and there is a possibility that the temperatures will change to the temperature setting of the heat source (target temperature+offset temperature).

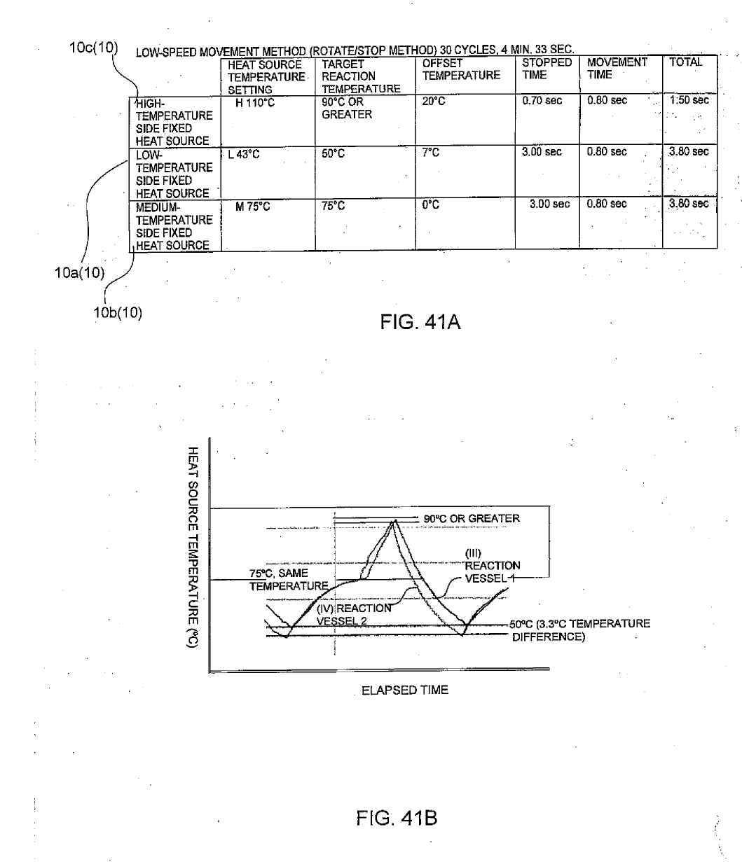

As an example, a test example is shown in FIGS. 41A and 41B in which the time to move to each heat source is fixed at 0.8 seconds, and offset temperatures are set for the fixed heat source 10a on the low-temperature side and the fixed heat source 10c on the high-temperature side. FIG. 41A shows the temperature settings for each heat source and the time to move to each heat source. As shown in FIG. 41A, the movement speed (time) for moving between each heat source is fixed, so the fixed heat source 10a on the low-temperature side and the fixed heat source 10c on the high-temperature side both are affected by the dynamic heat movement. Therefore, as shown in FIG. 41B, in a graph that shows the result of temperature control using this kind of construction, a temperature difference occurs in the low-temperature bottom (50° C. or greater) and high-temperature peak (90° C. or greater) between the back side and the front side in the direction of rotation.

Lines (III) and (IV) in this graph both show the temperature profile (temperature is along the vertical axis, and elapsed time is along the horizontal axis) of the reaction vessel 12 that is located on the back side in the direction of rotation (III), and the reaction vessel 12 that is located on the front side in the direction of rotation (IV) when the container 11 is stopped at a specified fixed heat source.

In order to solve this problem, the inventors of the present invention took the following measures. That is, taking into consideration the dynamic heat movement, it is considered that by changing the time that each well is in contact with the fixed heat source 10a on the low-temperature side, the amount of heat received by each portion (reaction vessels 12) of the container 11 can be made the same.

As long as the container 11 moves at a constant speed, stops, then moves at a constant speed to the next heat source, each reaction vessel 12 is located on the same container 11, so they come in contact with a heat source for the same amount of time. Therefore, when there is a dynamic temperature difference in the heat source, the effect of the dynamic temperature difference of the heat source on the heat received by each reaction vessel 12 cannot be avoided.

Therefore, in order to change the time during which each of the reaction vessels 12 that are located on the same container 11 come in contact with a specific heat source, the inventors of the present invention changed the speed at which a specified portion of the container 11 entered and left that heat source.

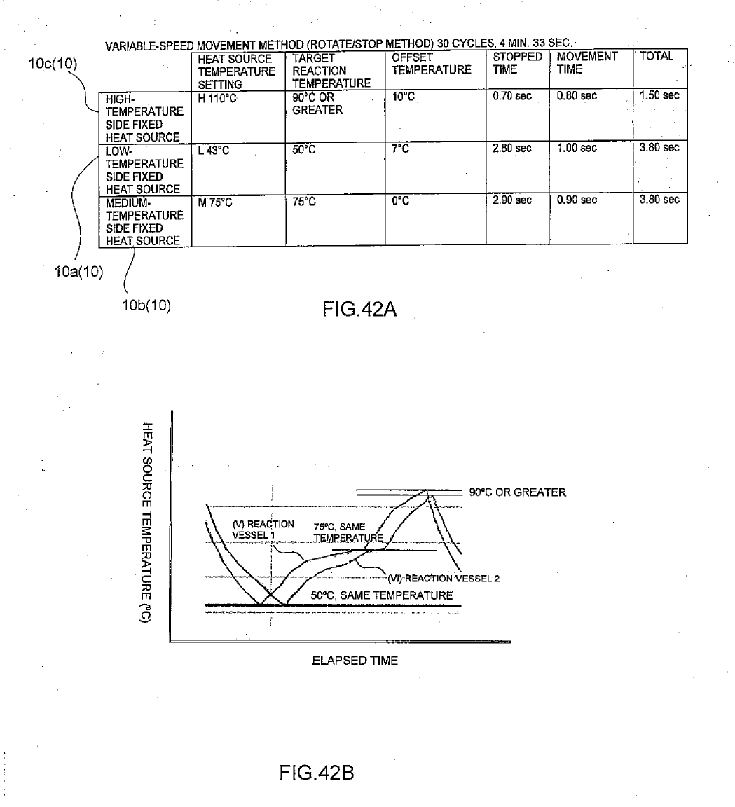

An example is explained with reference to FIGS. 42A and 42B.

In this example, the temperature settings of the fixed heat source 10a on the low-temperature side to the fixed heat source 10c on the high-temperature side, the amount of time that the container 11 is stopped at each of the heat sources, and the movement time between heat sources are set as shown in FIG. 42A. In other words, first, in order to set the target reaction temperature on the high-temperature side to 90° C. or greater, the temperature of the fixed heat source 10c on the high-temperature side is set to 110° C. so that it is offset from the target temperature by 20° C. In addition, the amount of time stopped at the fixed heat source 10c on the high-temperature side is set to 0.7 seconds, and the amount of time to move from the fixed heat source 10b on the medium-temperature side to this fixed heat source 10c on the high-temperature side is set to 0.8 seconds.

Moreover, in order to set the target reaction temperature on the low-temperature side to 50° C. or greater, the temperature of the fixed heat source 10a on the low-temperature side is set to 43° C. so that it is offset from the target temperature by 7° C. In addition, the amount of time stopped at the fixed heat source 10a on the low-temperature side is set to 2.80 seconds, and the amount of time to move from the fixed heat source 10c on the high-temperature side to this fixed heat source 10a on the low-temperature side is set to 1.0 second.

On the other hand, without providing an offset temperature for the fixed heat source 10 on the medium-temperature side, the amount of time stopped at the fixed heat source 10b is set to 2.90 seconds, and the amount of time to move from the fixed heat source 10a on the low-temperature side to this fixed heat source 10b on the medium-temperature side is set to 0.90 seconds.

In other words, the speed of movement is changed, so that even though the time to move from the fixed heat source 10b on the medium-temperature side to the fixed heat source 10c on the high-temperature side is set to 0.8 seconds, the time to move from the fixed heat source 10c on the high-temperature side to the fixed heat source 10a on the low-temperature side is set to 1.0 second, which is 0.2 seconds longer, and the time to move from the fixed heat source 10a on the low-temperature side to the fixed heat source 10b on the medium-temperature side is set to 0.9 seconds, which is 0.1 second longer. In this embodiment, measures are taken in this way to eliminate or minimize the temperature difference described above.

FIG. 42B is a graph that shows the result of temperature control from this kind of construction.

Lines (V) and (VI) in this graph show the temperature profile (temperature is along the vertical axis and time is along the horizontal axis) for reaction vessels 12 that are located on the back side in the direction of rotation (V), and for reaction vessels 12 that are located on the front side in the direction of rotation (VI) when the container 11 stops at a specific fixed heat source.

As shown in this graph, even though the offset temperature is set to 7° C. on the low-temperature side, two reaction vessels 12 both reached the same bottom temperature of 50° C. Moreover, at the medium temperature as well, the temperature of the two reaction vessels 12 were both controlled at 75° C. On the other hand, on the high-temperature side, a temperature difference occurred between the two reaction vessels 12 at the target temperature of 90° C. or greater, however, in a reaction that separates two strands of DNA by heat, there is no problem with the reaction when the high temperature is 90° C. or greater even when there is a temperature difference. This embodiment takes advantage of this by setting the movement time such that both the low temperature and medium temperature become the set temperature.

In other words, generally, even by taking note that there is a temperature difference between each of the heat sources and changing the movement speed, the reaction vessels are on the same container, so even though the movement speed is changed for one heat source, that change is the same for other portions as well. That is, by considering the effect of the dynamic heat movement of the first and second heat source, that effect can be eliminated by adjusting the speed; however, the third heat source cannot be set to similarly avoid the effect. Of the three temperatures necessary for PCR, the low temperature and medium temperature must be fixed temperatures, however, in a reaction that separates two strands of DNA by heat, there is no problem as long as the high temperature is 90° C. or greater even when there is a temperature difference.

A method of making the heating history of each reaction well the same, even though there may be dynamic temperature changes in the heat sources, can also be achieved by a method of continuously rotating the container at constant speed.

(Enzyme Speed)

The framework of the present invention for making nucleic acid amplification fast was described above, however, besides this there are things that must be kept in mind. One in particular that should be mentioned is that base elongation occurs at the aforementioned medium temperature, and when the thermal cycle is set to a high speed, the target amplification may not be performed when the base elongation capability of the polymerase that is used is not sufficient. Taq, which is generally used in PCR, has a capability of 50 bases/second, and may be insufficient in high-speed PCR of 5 minutes or less. Therefore, it is preferred that a polymerase having the capability of 100 bases/second or greater, such as Takara Z-Taq or Toyobo KOD-Dash, be used.

(Fluorescent Light Measurement Device)

FIG. 13 schematically shows the relationship between a fluorescent light measurement device 27 and a reaction vessel 12 that is the object of measurement. This fluorescent light measurement device 27 comprises an excitation light generation unit 45, reflection unit 46 and photomultiplier tube unit 47. The excitation light generation unit 45 is arranged such that an internal LED excitation light source 48 is facing upward, and shines the generated excitation light onto the liquid reaction mixture in the reaction container 11 that is on top via an excitation light filter 49. The reflection unit 46, by way of a mirror 50, reflects the fluorescent light that is emitted from the liquid reaction mixture toward the photomultiplier tube unit 47, and adjusts the light to the required wavelength by way of a fluorescent filter 51. The photomultiplier tube unit 47 measures the fluorescent light that is directed to it via the reflection unit 46.

This fluorescent light measurement device 27 is held by a movable table 28 that moves along the radial direction of the reaction container 11, and when not in use is located away from the reaction container 11 such that only when it is used to measure fluorescent light it moves to a position that faces the reaction vessels 12 and performs measurement. As shown in FIG. 14, this fluorescent light measurement unit 27 and movable table 28 are located so that they correspond with a space that is formed between the fixed heat source 10a on the low-temperature side and the fixed heat source 10b on the medium-temperature side; and being synchronized with the timing that the reaction vessels 12 come to this space, the fluorescent light measurement unit 27 executes measurement of each reaction vessel 12.

The measurement of fluorescent light can also perform so-called real-time PCR by watching the change over time of fluorescent light at a fixed temperature, and by watching for the temperature (offset temperature) at which the fluorescent light disappears as the temperature is slowly raised after nucleic acid amplification, the amplified nucleic acid can be identified.

In fluorescent light measurement, there is penetrating type and reflective type, however, because the measurement sensitivity is also improved, reflective type, as in this embodiment, is preferred. In this embodiment, in order to improve the reflection efficiency, a metallic reflective film 54 is attached to the inside surface of the reaction vessels 12 as well, and the base plate 37 of the reaction container 11 or the blocking member 39 on the upper side can be formed from a light-shielding material. On the other hand, film 38 on the bottom surface side is partially or completely translucent.

(Operation and Control of the Device)

Next, the control and operation of this device will be explained.

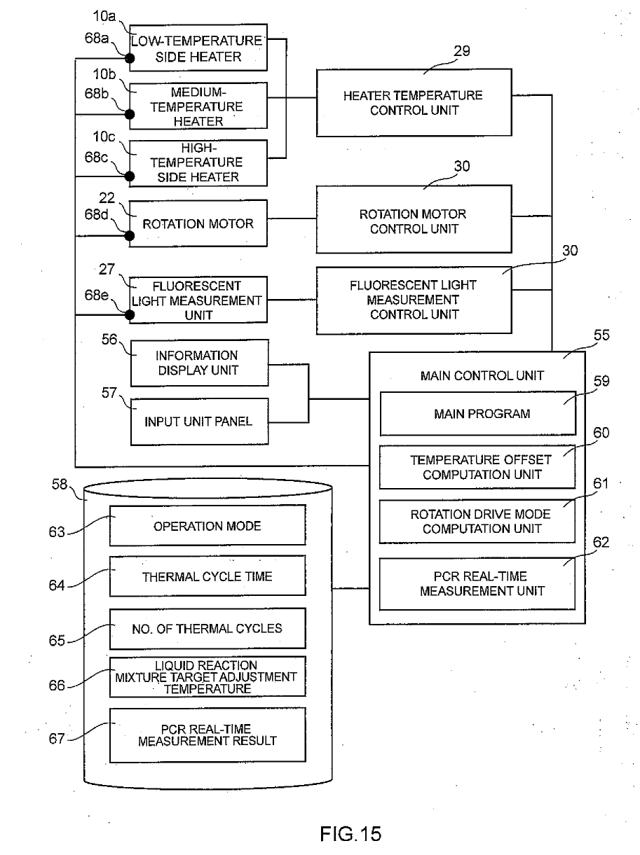

FIG. 15 is a block diagram showing the control system for this device.

The same reference numbers will be given to component elements that have already been explained, and an explanation of them will be omitted. Of the components shown in this block diagram, a main control unit 55, information display unit 56, input unit 57 and memory unit 58 are actually constructed using a computer, and are included below the front panel 54 of the device shown in FIG. 4.

The information display unit 56 is more specifically a liquid-crystal panel, and the input unit 57 is a touch sensor or key input unit that is provided on this liquid-crystal panel. The main control unit 55 is an operating device comprising a CPU and RAM, and has a main program 59, temperature offset computation unit 60, rotation drive mode computation unit 61 and real-time PCR measurement processing unit 62. The main program 59 displays information input requests needed for control to an operator, and the information that is inputted by the operator is stored in the memory unit 58 that comprises a hard disk or the like. In other words, this memory unit 58 stores the operation mode 63, thermal cycle time 64, number of thermal cycle repetitions 65, target adjustment temperature for the liquid reaction mixture 66 and PCR real-time measurement results 67.

The aforementioned heater temperature control unit 29, rotation motor control unit 30 and fluorescent light measurement control unit 31 are connected to the main control unit 55. In addition, sensors 68a to 68b that are provided in the heat sources 10a to 10c are also connected to the main control unit 55, and the main control unit 55 is such that feedback control is set to each component element based on detected values from the sensors.

(Operation Flow)

The operation of the device will be explained below by following a flowchart. The reference codes S1 to S17 in the figure correspond to the following steps S1 to S17.

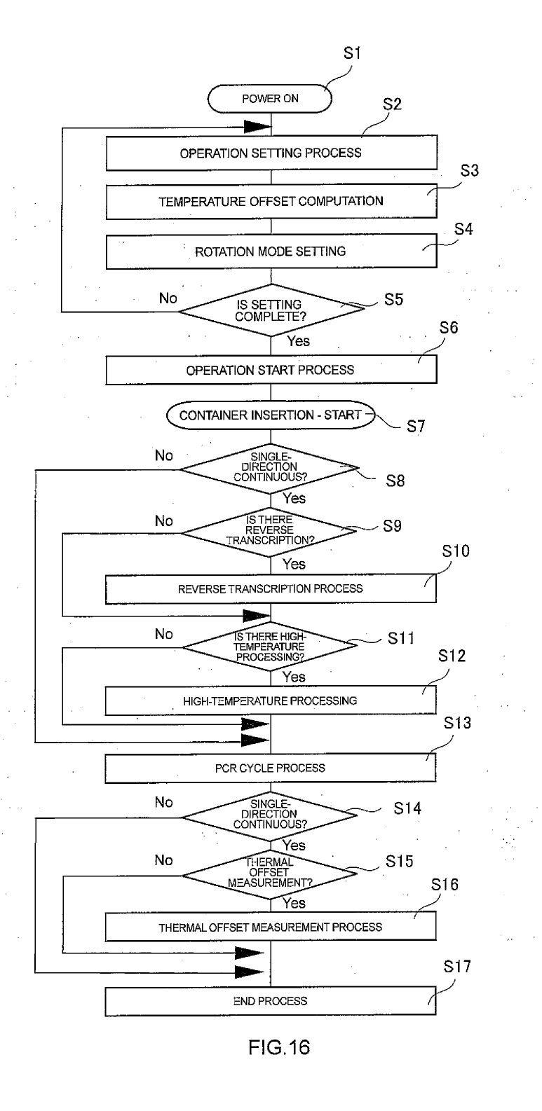

FIG. 16 is an operation flowchart showing the operating procedure.

First, after the power is turned ON (step S1), the operation mode is set in an operation setting process step (step S2). This device is such that by when selecting the operation mode, in addition to selecting just the PCR reaction, it is possible to select whether or not to perform a reverse transcription reaction, whether or not to perform a hot start and whether or not to perform thermal offset measurement. However, these are always performed in real-time fluorescent light measurement.

In this operation setting process (step S2), in addition to the reaction mode, the thermal cycle time, number of thermal cycle repetitions and target adjustment temperature for the liquid reaction mixture are set. The input for setting these is performed interactively via the information display unit 56 and the input unit panel 57, and the main control unit 65 stores the inputted information in the memory unit 58 (FIG. 15).

After the operation setting process is finished, this device is such that the temperature offset computation unit 60 computes the necessary offset temperature, and the rotation drive mode computation unit 61 computes and sets a suitable operation mode such as the movement speed to each temperature heat source, or the amount of time stopped above each heat source.

After setting is finished (step S5), operation begins, and the peak temperature control unit 29 drives and raises the temperature of the heat sources 10a to 10c. When the heat sources 10a to 10c reach the specified temperatures and preparation is complete, the top cover 13 is opened, the reaction container 11 is inserted and the cover is closed, after which a start instruction is given from the input unit panel 57. After that, the required reaction process is sequentially performed as will be explained below.

First, whether operation is single-direction continuous operation is determined (step S8). When single-direction continuous operation is selected, only the PCR reaction process is performed.

When operation is something other than single-direction continuous operation, then next whether there is a reverse transcription process is determined (step S9), and when there is a reverse transcription process, the reverse transcription processing is executed before the PCR cycle (step S10). In this process, first, the heat source 10a on the low-temperature side is set to the reverse transcription temperature and maintained for a specified amount of time. After that, high-temperature processing is performed at the heat source 10c on the high-temperature side to deactivate the reverse transcriptase enzyme.

Next, when this device performs hot-start PCR, whether or not there is high-temperature processing is determined (step S11), and after being maintained at the heat source 10c on the high-temperature side for a specified amount of time before the PCR cycle (high-temperature process: step S12), operation advances to the PCR cycle (step S13 and later).

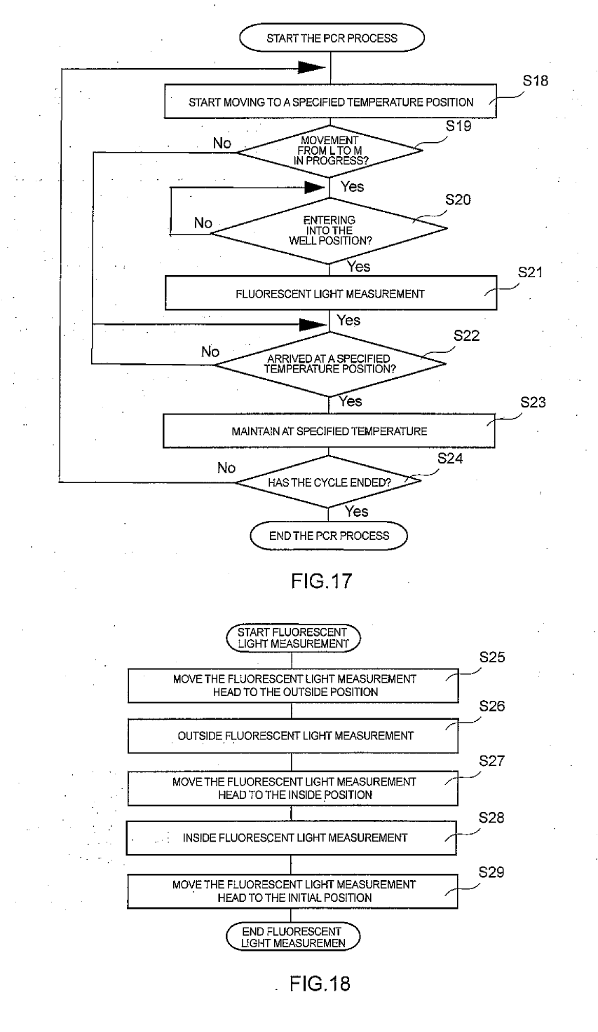

Next, the PCR process of step S13 is executed. This PCR process is shown in the flowchart of FIG. 17. By executing each step (S18 to S24) in FIG. 17 in order, the temperature of the heat source on the high-temperature side TH1, the temperature of the heat source on the low-temperature side TL1 and the temperature of the heat source on the medium-temperature side that were calculated by the temperature offset computation unit 60, and the amount of time at each heat source that was set be the rotation drive mode computation unit 61 can be repeated a set number of cycles, and while each of the reaction vessels 12 move in the space 52 between the heat source 10a on the low-temperature side and the heat source 10b on the medium-temperature side, the fluorescent light measurement device 27 can measure the fluorescent light from the liquid reaction mixture.

These measurement values are sequentially stored in the memory unit. It is also possible, for example, to transfer the values to another computer in real-time via a network.

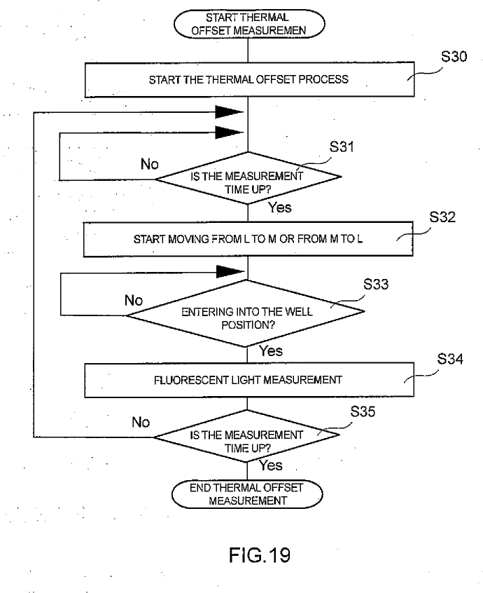

In this process, it is possible to perform just the PCR process without measuring the fluorescent light in real-time. In that case, the thermal offset measurement process can be executed in step S16 later. FIG. 19 is a flowchart showing this thermal offset measurement process.

When this process begins (step S30), first, at the end of PCR, operation stops at the position of the heat source 10a on the low-temperature side and waits for a specified amount of time until the heat source 10a on the low-temperature side and the medium-temperature heat source 10b reach the thermal offset starting temperatures, after which operation waits 30 seconds (step S31). After that, the temperatures of both heat sources are raised at the set speed for raising the temperature, and after every set interval of a few seconds, the fluorescent light is measured while moving the reaction vessels between the heat source on the low-temperature side and the medium-temperature heat source (steps S32 to S35).

This device can also be applied to a reaction container other than the container having the shape explained in the embodiment described above.

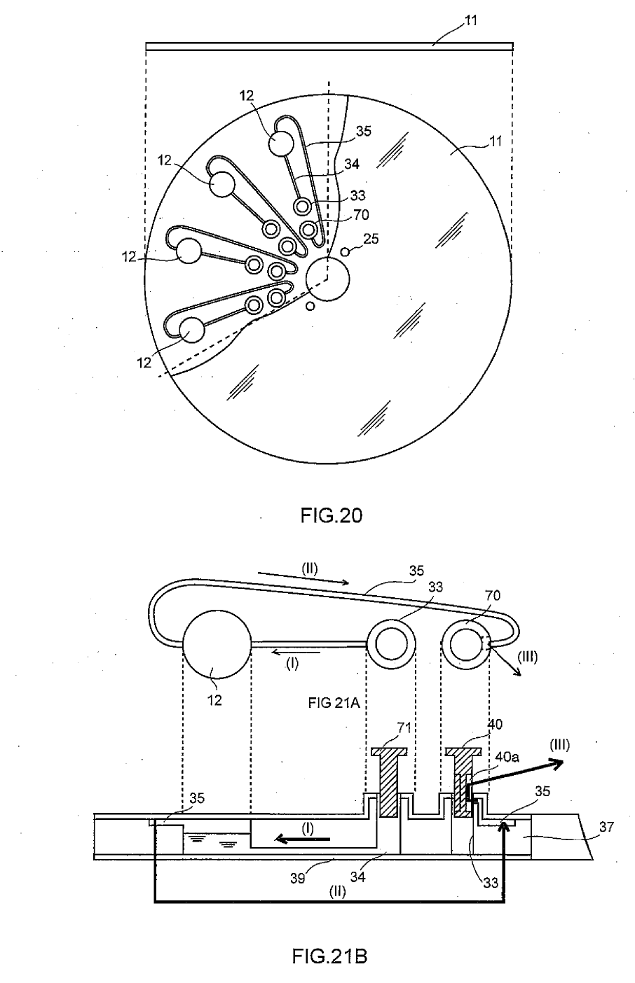

For example, in the container shown in FIG. 6, FIG. 8A and FIG. 8B, in order to pressurize and seal the inside of the reaction vessels so that the pressure inside them is greater than the atmospheric pressure around them, one liquid feed hole and one seal stopper were used, however, as shown in FIG. 20, FIG. 21A and FIG. 21B, it is possible to independently provide a liquid feed hole 33 and air removal hole 70, and to insert a seal stopper 40, 71 in each, respectively. With this kind of construction, more reliable operation can be obtained, and by using two seal stoppers 40, 71, the freedom of controlling the internal pressure is increased.

A groove 40a is formed on a portion of the side surface of the stopper 40 shown in FIG. 8B. When the liquid reaction mixture is fed through the liquid feed hole and the stopper 40 is inserted and pressed down, the portion from the bottom of the stopper 40 to the groove 40a is sealed by the liquid feed hole wall 33, so the liquid reaction mixture is fed into the reaction vessel 12 from the liquid feed hole as the stopper is pressed down.

In order to prevent the internal pressure from increasing while the liquid is being fed in, the air inside the reaction vessel 12 is allowed to pass through the upper portion 33a of the liquid feed hole 33 and to escape to the outside through the groove 40a. As the stopper 40 continues to be pressed after that, the groove 40a finally covers the air hole 33a from the groove 35, and by pressing the stopper 40 to the very bottom, feeding of the liquid can be completed and then a fixed internal pressure can be applied.

FIG. 21B shows an example of increasing the freedom of the design of the groove 40a by stopping the liquid feed and controlling internal pressure using separate stoppers.

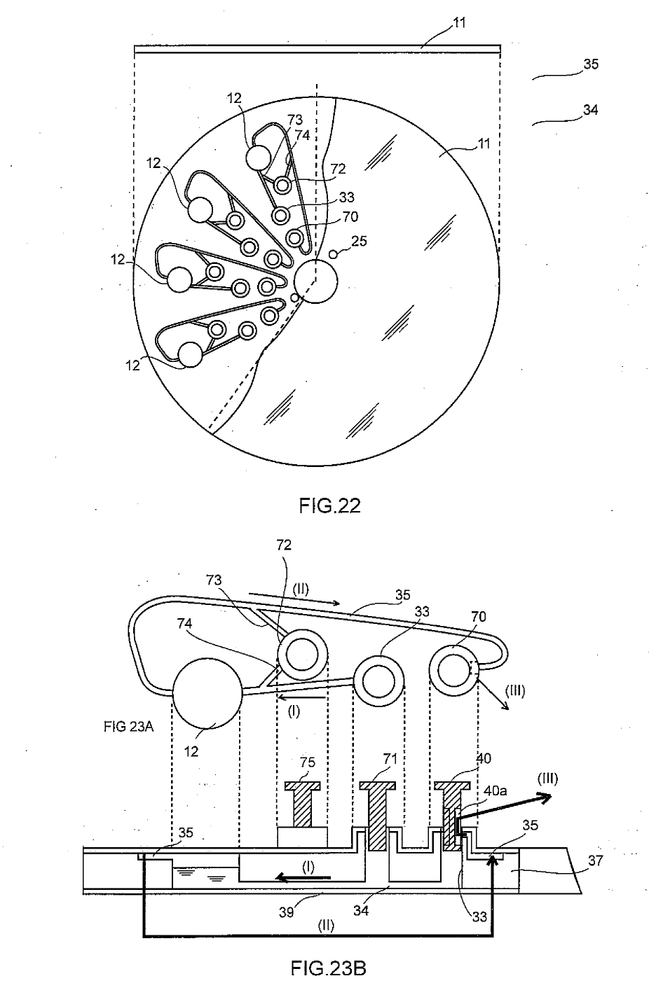

Moreover, as shown in FIG. 22, FIG. 23A and FIG. 23B it is also possible to provide a liquid feed channel 34 and a sealant inlet hole 72 for putting sealant in the atmospheric pressure release channel 35 in addition to the container shown in FIG. 6. With this kind of construction, by pressing a seal stopper 75 into this sealant inlet hole 72, sealant that is inside the sealant inlet hole 72 can be filled into the liquid feed channel 34 and atmospheric pressure release channel 35 by way of channels 73, 74. This sealant is a material that hardens under certain conditions. With this sealant, it is possible to further increase the independence and seal of each of the reaction vessels 12.

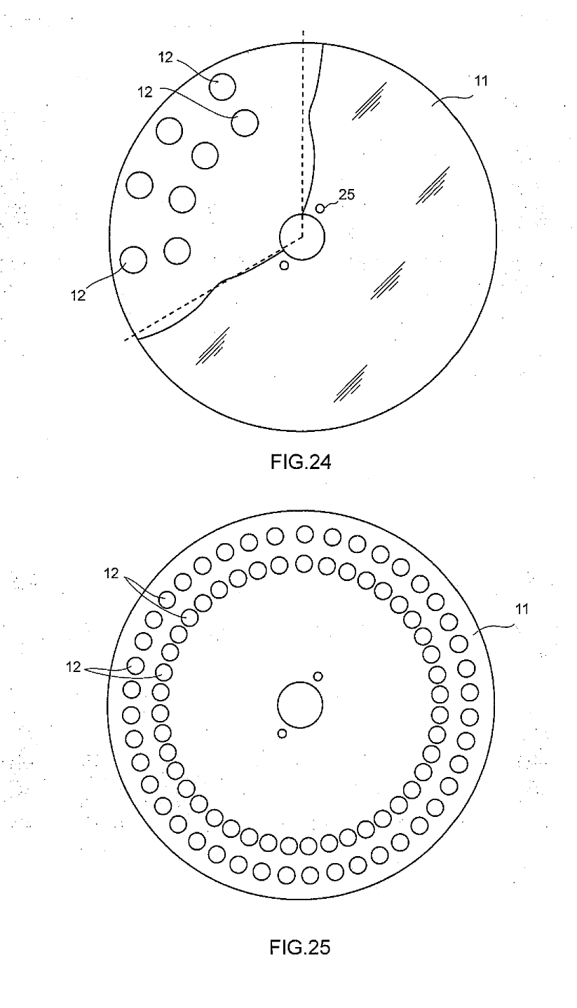

Also, in the reaction container 11 shown in FIG. 6, FIG. 20 and FIG. 21, the top surface and bottom surface of the reaction vessels 12 are blocked beforehand, and the liquid reaction mixture was filled into the reaction vessels 12 from the liquid feed hole 33 and through the liquid feed path 34, however, the invention is not limited to this. For example, it is possible to directly fill the liquid reaction mixture directly into the reaction vessels 12 without using a liquid feed hole 33. The example shown in FIG. 24 is an example in which eight reaction vessels 12 are independently provided such that they are separated from each other. In this case, for example, the top surface blocking plate 38 is made of a flexible sheet, and by attaching this sheet to the top surface of the main substrate 37 it is possible to block together all of the reaction vessels 12 that are filled with liquid reaction mixture.

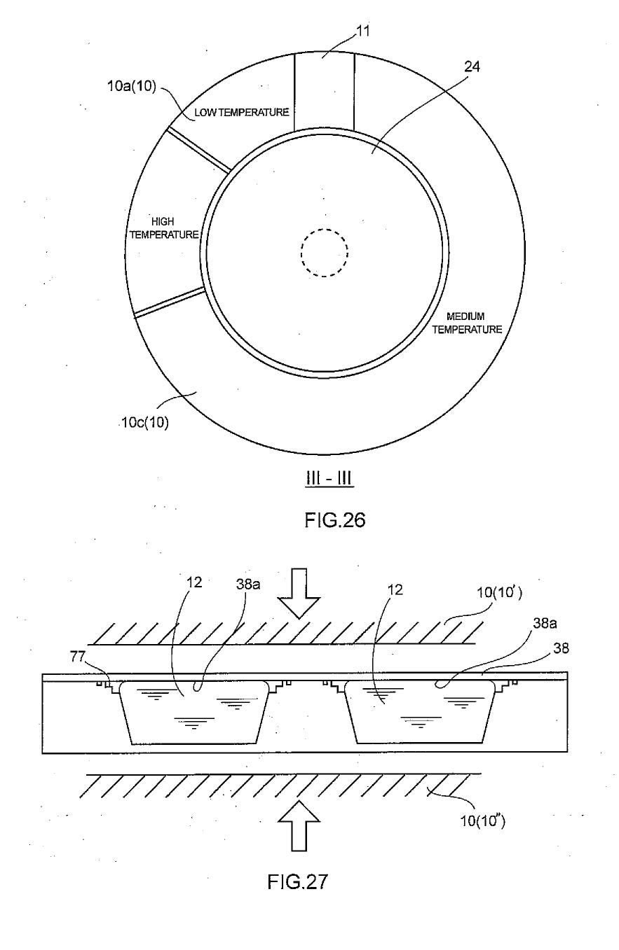

Furthermore, as shown in FIG. 25, the reaction vessels 12 can be provided all around the entire circumference. In this case, the container can be used for testing a large number of specimens. In the case of the reaction container 11 shown in FIG. 25, by rotating the container 11 continuously in a single direction, the thermal cycles can be applied to each of the reaction vessels 12. When the container 11 is rotated continuously in a single direction in this way, only the first thermal cycle in the reaction space has three types; one that starts from high temperature, one that starts from low temperature and one that starts from medium temperature, and by properly selecting a PCR primer, the difference will not become a problem, so it is possible to process a large number of specimens at one time. Another example of an arrangement of heat sources that is suitable to the reaction container shown in FIG. 25 is shown in FIG. 26. That is, the area ratios of the heat sources 10a to 10c are arbitrary, and it is possible to locate them at 120 degree intervals as in the embodiment described above; however, as shown in this figure, by making the area of each heat source equal to the temperature retention time ratios of the heat sources (for example, heat source on the high-temperature side:heat source on the low-temperature side:medium-temperature heat source=1:1:4), PCR can be performed using 30 cycles at 3 minutes each for multiple specimens.

An enlarged view of the reaction vessel 12 portion of this kind of reaction container is shown in FIG. 27.

In the case of a multi-specimen container, each reaction vessel 12 is relatively small, so when applying the blocking member 38 on the surface after putting the liquid reaction mixture inside, the liquid reaction mixture may spill onto the top surface of the substrate 37 of the container making it difficult to perform the adhesion. After struggling to find a countermeasure to this by trying various methods, the inventors of the present invention solved this problem by providing a 0.1 mm high stepped section 77 around the circumference of the top surface of the reaction space as shown in FIG. 27, and by performing hydrophobic processing on that area. Preferably, hydrophobic processing is also performed on the bottom surface of the film 38 of the surface blocking member (portion indicated as 38a in the figure). By having a stepped section 77, and by further making that area hydrophobic, air remains in a ring shape around the outside of the reaction vessels 12 when the liquid reaction mixture is pressed by the film 38 (39), thus it is possible to prevent liquid reaction mixture from seeping onto the adhesion surface. This makes it possible to eliminate failure when applying the film.

The measure for preventing leakage of liquid is extremely important, and from a practical point of view, is essential for independent containers on which surface blocking film is applied after the liquid has been put into the independent reaction spaces all around the circumference as shown in FIG. 25. That is because in that case, liquid reaction mixture is put into 60 reaction spaces at one time, and since all of the independent spaces are close to each other, the results lose reliability even when just a small amount is leaked. This is because, depending on the target of the test, the result could be fatal, or could be life threatening in the case that leaked fluid is touched.

When performing fluorescent light measurement using a multi-specimen reaction type container as shown in FIG. 24 or FIG. 25, it is necessary that the fluorescent light device be faced toward each reaction vessel. The control of the fluorescent light measurement device 27 in this case is shown in the flowchart of FIG. 18. In other words, as shown in FIG. 18, in steps S25 to S29, the fluorescent light measurement head is moved back and forth in the radial direction of the reaction container 11, making it possible to sequentially face the reaction vessels 12 on the outer circumferential side and the reaction vessels 12 on the inside to perform fluorescent light measurement.

### Second Embodiment

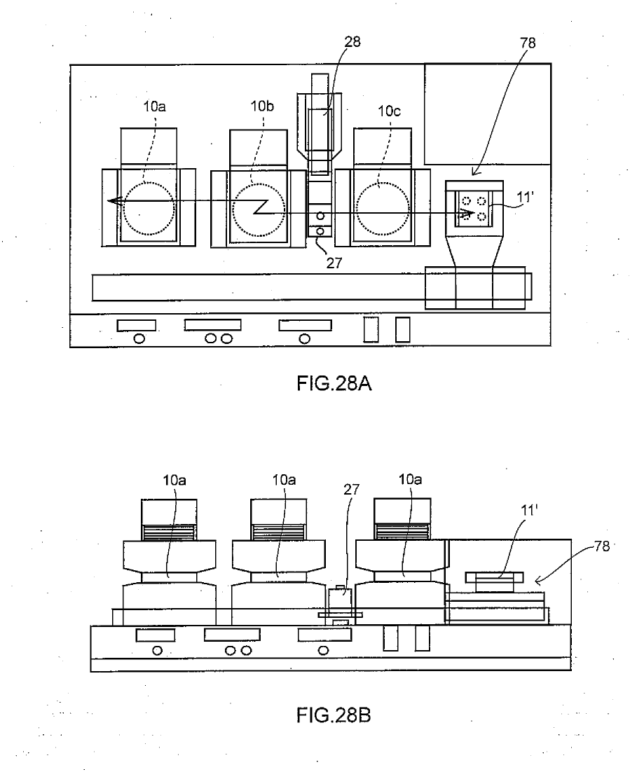

Next, a second embodiment of the present invention will be explained with reference to FIG. 28 to FIG. 30.

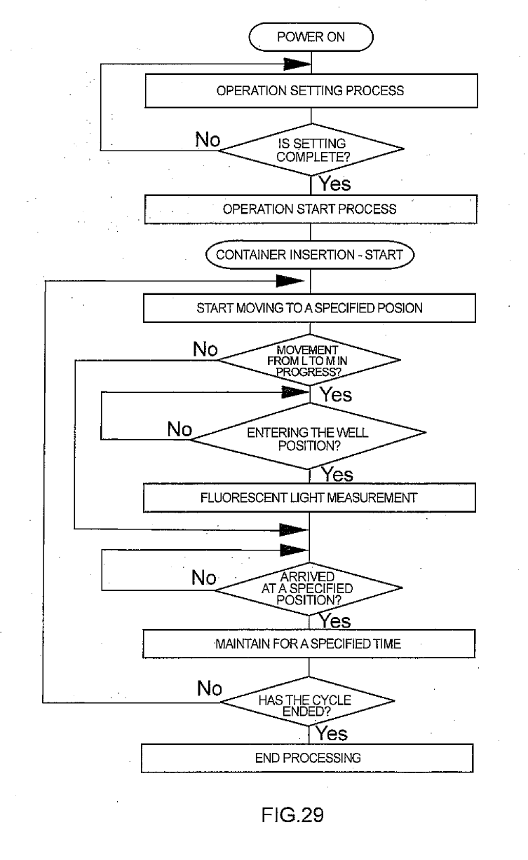

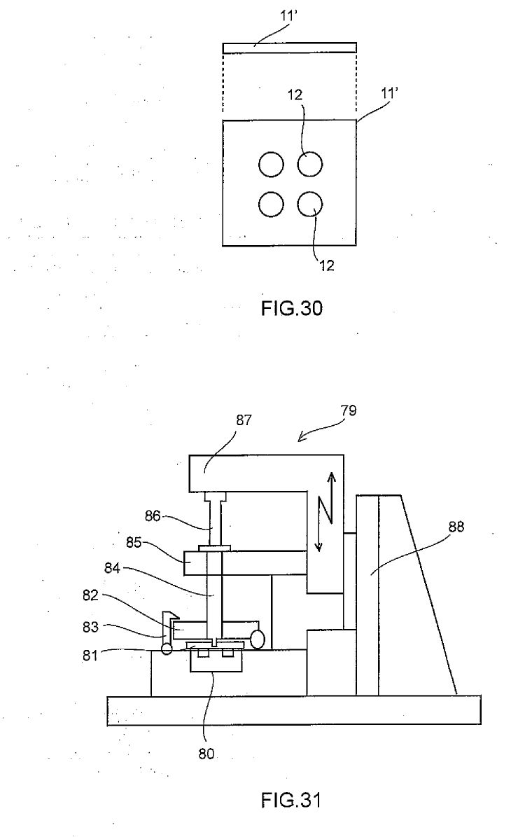

FIG. 28A is a top view of the device of this second embodiment, FIG. 28B is a front view, FIG. 29 is an operation flowchart, and FIG. 30 shows a rectangular flat shaped reaction container 11′ that is used in this device.

In other words, in the first embodiment described above, the reaction and measurement were performed by using a circular plate shaped reaction container and rotating it, however, the device of this second embodiment performs the same operation as the device of the first embodiment by moving in a single direction without rotating the reaction container of FIG. 30.

The theory and operation are the same as in the first embodiment, however, this embodiment differs from the first embodiment in that instead of the fixed heat sources being arranged around the circumference, they are arranged in a straight line.

The fluorescent light measurement device 27 is also the same mechanism as in the first embodiment, however it is located between the heat source units 10a and 10b. A container holder 78 is provided next to the heat sources 10a to 10c, and is such that the PCR reaction is performed by holding the rectangular flat shaped reaction container 11′ and moving it back and forth along a guide as shown by the arrow 79 in the figure. The container holder 78 is constructed such that by inserting the reaction container 11′ into a groove (not shown in the figure) and fastening it with a container stopper, the container 11′ is prevented from being dropped during movement.

The PCR reaction processing procedure and control method for this device is shown in the flowchart of FIG. 29, however, except for the container moving in the straight line, these are the same as in the first embodiment, so an explanation thereof is omitted.

### Third Embodiment

Next, a third embodiment of the present invention will be explained with reference to FIG. 31 to FIG. 36.

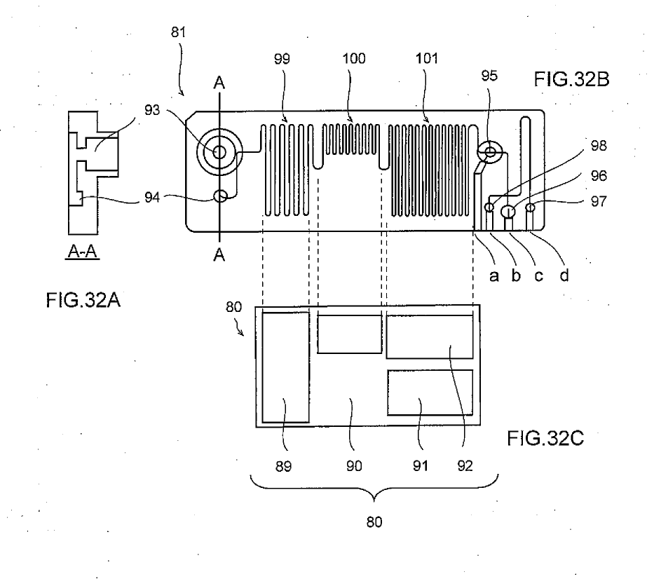

FIG. 30 is a side view showing the device 79, FIG. 32 shows the reaction container 81 that is used in this device, and FIG. 32 is a top view showing the heat source 80 that corresponds to the reaction container.

The first and second embodiments applied thermal cycles to the liquid reaction mixture by moving the reaction container 81 with respect to the heat source 80, however, in this embodiment, thermal cycles are applied to the liquid reaction mixture by keeping the positional relationship between the reaction container and the heat source fixed, and moving the liquid reaction mixture inside a flow channel that is provided in the reaction container.

In other words, as shown in FIG. 32B, the flow path container is a 2.51 mm thick rectangular flat plate that is constructed by adhering a 10 μm thick polycarbonate film to a 2.5 mm molded polycarbonate main section. A liquid inlet 93, in which a syringe filled with liquid reaction mixture is inserted, is provided on one end section of the container, and the flow channel groove that leads out from this liquid inlet 93 extends to a PCR liquid arrival observation window 96 that is provided on the other end section of the reaction container 81. This flow channel groove is a 200 μm wide and 50 μm deep flow channel groove, and is partitioned and constructed as a flow channel for the liquid reaction mixture by blocking the opening on the top end by the polycarbonate film described above.

The liquid reaction mixture flows through this flow channel groove from the liquid inlet 93 toward the PCR liquid arrival observation window 96 such that the PCR reaction and observation are a series of processes. Here, the flow channel is formed such that it runs back and forth in an accordion shaped path in the width direction of the container as shown in FIG. 32, and in so doing, forms three areas, or in other words, a reverse transcription area 99, enzyme deactivation area 100 and PCR area 101.

An electrophoresis liquid inlet 97 for performing electrophoresis and an electrophoresis liquid arrival observation opening 98 that are separated from each other are provided on the other end section of the reaction container 81 from where the PCR liquid arrival observation window 96 is provided, and these are connected by a different flow channel groove than that described above. Moreover, as shown in the figure, the two flow channel grooves are connected in a crossed shape near the electrophoresis liquid arrival observation opening 98. Furthermore, liquid arrival observation openings 94, 95 are provided on the other end section of the reaction container 81. The liquid arrival observation openings 94, 95 are for visually observing where the liquids inside the flow channels have reached, and become liquid reservoirs inside the flow channels. Also, electrodes a to d are provided on the end surface of the flow channel container, and these electrodes are exposed to one side second of the container. The electrodes a to d are constructed such that they are wired inside the container 81 to the liquid arrival observation opening a94, electrophoresis liquid observation opening 98, PCR liquid arrival observation window 96 and electrophoresis inlet 97, respectively, and come in contact with the liquid.

In the explanation of the operation given below, in order to simplify the explanation, the notation, window A=liquid inlet, window B=liquid arrival observation opening a94, window C=liquid arrival observation opening b95, window D=PCR liquid arrival observation window 96, window E=electrophoresis liquid arrival observation opening 98, and window F=electrophoresis liquid inlet 97 is used.

On the other hand, FIG. 32C shows the heat source 80 for applying heat to the reaction container 81. This heat source 80 is located directly below the flow channel container 81 and comprises heat source sections for reverse transcription 89, for enzyme deactivation 90, for PCR high temperature 91 and PCR low temperature 92, which correspond to each site of the reaction container. The temperatures of the heat source sections 89 to 92 are controlled by a temperature controller to temperatures that correspond to the respective reactions.

As shown in FIG. 31, this device is constructed such that the reaction container 81 is located above the heat source 80, and drives a pressure cover 82 and heat source by way of a container lock in a direction such that they press against each other. A syringe 84 that is filled with the liquid reaction mixture is set in a syringe support block 85 and inserted through a hole that is opened into the pressure cover 82 into a syringe insert opening in the flow channel container 81, then by pressing the piston section 86 of the syringe down by way of a piston push-down block 87, the liquid reaction mixture is fed into the container. The piston push-down block 87 is moved up or down by a stepping motor (not shown in the figure) that is provided with a single-axis moving table 88. The speed of feeding the liquid, or in other words, the speed of the stepping motor is controlled by a microcomputer inside the control unit.

In regards to the electrophoresis, a prescribed voltage is generated by a electrophoresis heat source, and electrodes on the side of the device (not shown in the figure) are located on the side of the heat source in positions that correspond with the electrodes a to d of the flow channel container, and are constructed such that when the pressure cover 82 is locked, the container and electrodes on the side of the device come in contact and power flows.

A fluorescent light detection mechanism for seeing the results of electrophoresis is not assembled in the machine of this embodiment, and measurement is performed externally by inserting a USB4000 spectrometer made by BAS Inc., and a reflected light measurement probe that is connected to a light source into a hole in the fluorescent light measurement unit. Other than this, the device is constructed such that it has a control unit for performing overall control and an operation unit.

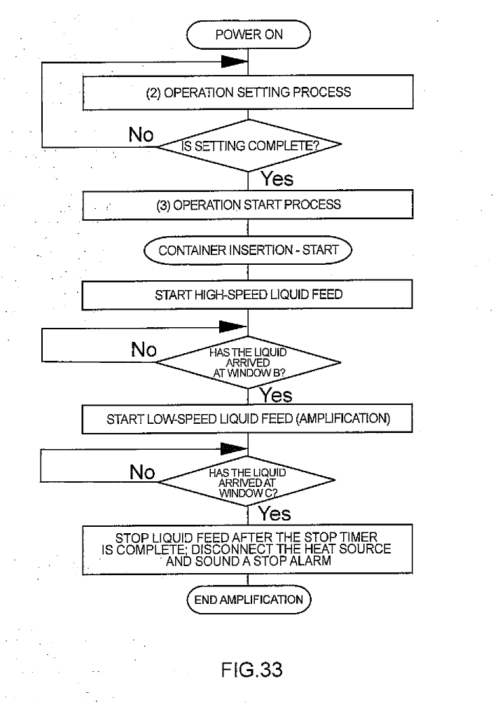

FIG. 33 to FIG. 35 are flowcharts showing the operation of this device.

FIG. 33 is a flowchart showing the main PCR reaction process.

First, when starting a PCR reaction or the like in the reaction container of this device, the reaction container is set as described above, and after the restraining cover 82 is locked, the syringe 84 filled with the liquid reaction mixture is set and the start button is pressed. By doing so, the piston push-down block 87 is lowered at high speed until the liquid reaction mixture reaches window A. The arrival of the liquid at window A is determined by a microcomputer in the operation unit according to a liquid feed time that was determined through experimentation beforehand. After that, the piston push-down block 87 continues to be lowered at low speed so that a specified liquid feed speed is obtained. After an amount of time has elapsed for the liquid to probably reach window D, feeding of the liquid is stopped, and the operator is notified of the end of the reaction by a buzzer.

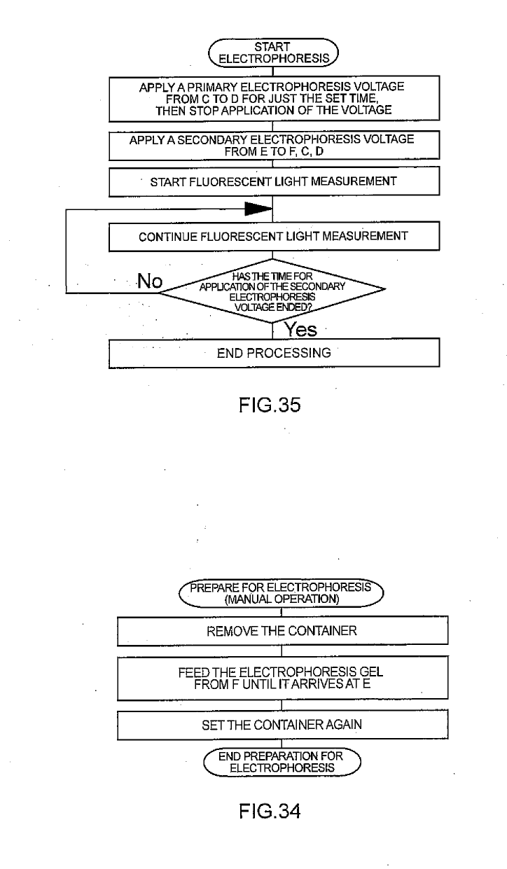

Next, as shown in FIG. 34, the flow channel container 81 is removed, and Agarose gel for electrophoresis is injected from window F until it can be seen at window E, then the flow channel container 81 is set into the flow channel device. When doing this, the syringe is left removed so that the liquid reaction mixture does not move any more than this. As shown in FIG. 35, next the electrophoresis start button is pressed, and after the primary electrophoresis voltage is applied from window C toward window D for a fixed amount of time, the voltage stops. Continuing, a secondary electrophoresis voltage begins to be applied from window E toward window F. After that, all processing is stopped after the set time for applying the secondary voltage has elapsed.

### Fourth Embodiment

Next, a fourth embodiment of the present invention will be explained.

The first and second embodiments moved the reaction container, and in the third embodiment construction was such that specified thermal cycles were applied to the liquid reaction mixture by moving the liquid reaction mixture, however, the device of this fourth embodiment is constructed such that neither the container nor the liquid reaction mixture is moved.

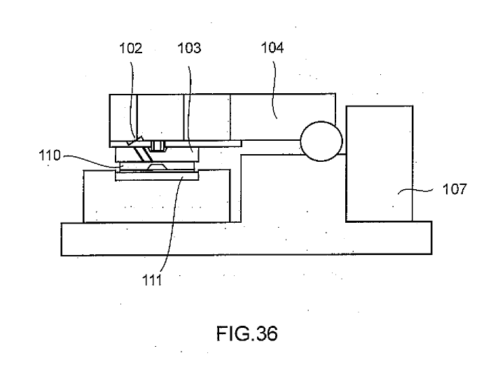

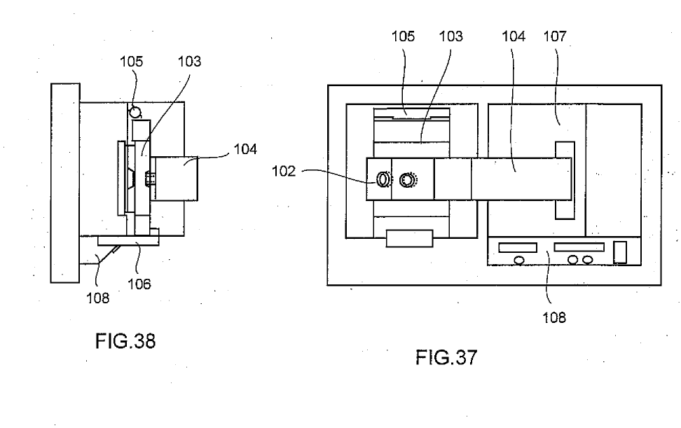

FIG. 36 is a side view of the device, FIG. 37 is a top view of the device, and FIG. 38 is a front view of the device.

In FIG. 36, 110 indicates the reaction container. The reaction container 110 has a shape as shown in FIG. 27 and independent reaction vessels 12 are formed on top. The reaction container 110 having this shape is turned upside down and located above the heat source 111.

In this device, the reaction applies a specified heat to the liquid reaction mixture that is filled in the fixed container 110, and construction is such that in the case of the PCR method, the progress of the reaction is checked by a fluorescent light device, and in the case of the LAMP method, the liquid reaction mixture is checked visually to determine whether it has become cloudy.

Heating and cooling are both performed by heat source 111 by just one Peltier element. The container 110 is placed above the heat source 111, and as shown in FIG. 37, a through hole is formed in order that the excitation light irradiation axis 102 is not obstructed, and a glass pressurization member 104 made of hard glass and made to be light shielding by a reflective metallic film covers the area around the hole, and with a locking mechanism 106, which is shown in the left cross-sectional drawing of the device construction, the heat source 11 and fixed container 110 are secured such that they are pressed against each other. The fluorescent light measurement unit 104 is upside down from the construction shown in FIG. 13, and instead of a moving table, is attached to the base with hinge construction 105 as shown in FIG. 37. After the reaction container 110 has been fastened by the transparent glass member, the hinge is used to lower the fluorescent light measurement unit 104 by hand until it is positioned above the transparent glass member. Other than this, the device is constructed with a control unit 107 that performs overall control, and an operation unit 108.

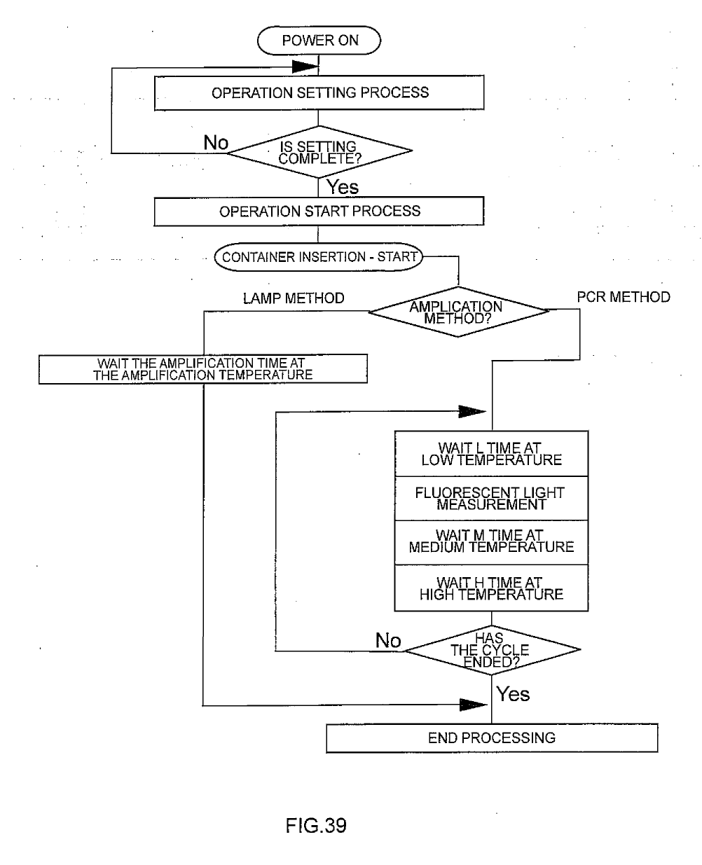

FIG. 39 is a flowchart of the operation flow.

As shown in this figure, basically, after the reaction conditions are set, the fixed container filled with liquid reaction mixture is set and the fluorescent light measurement unit is covered, the reaction is performed automatically by pressing the start button.

In the case of the LAMP method, the container is simply kept at a temperature that corresponds to a specified temperature for a specified amount of time, and after that, the reaction results are judged by whether or not the liquid reaction mixture is cloudy. In the case of the PCR method, a cycle of maintaining the container at specified temperatures by heat sources that correspond to high temperature→low temperature→medium temperature for a specified amount of time is repeated a specified number of times. At the end of the low temperature of each cycle, a fluorescent light measurement is performed to determine the progress of the reaction.

A first through a fourth embodiment of the invention were explained above, however, next, an example of the dimensions of the reaction container and the liquid reaction mixture used will be described.

(Reaction Container for a Circular Plate Device)

Here, the “reaction container for a circular plate device” is the reaction container that is used in the first embodiment.

The containers are each a circular disk that is formed from 0.6 mm thick polycarbonate and has a diameter of 120 mm, with a circular hole being formed in the center section that corresponds with the axis of rotation.

Hereafter, for convenience sake, the different shaped “reaction containers for a circular plate device” will be referred to as a “8-well container”, “entire circumference container”, and “a container, b container, c container”.

The “8-well container” is the container shown in FIG. 24, and is a flat plate that is entirely transparent, comprising two rows of four circular fan-shaped regions having a 120-degree outer circumference, for a total of eight circular concave sections. The bottom of each concave section has a thickness of 0.2 mm, with two 0.1 mm deep circular shaped stepped sections being provided on the top end surface, and together with being coated by a reflective metallic film, hydrophobic processing is performed for the stepped sections.

After liquid reaction mixture has been filled into each concave section, a 100 μm thick fluoropolymer resin film is adhered and the container is ready for the reaction.

The “entire circumference container” is the container shown in FIG. 25, and is the same as the 8-well container except that 60 concave sections are arranged around the entire circumference, and the color of the material for ensuring that each of the concave sections is light shielding is black.

The “a container, b container and c container” have the same basic construction as the 8-well container with a transparent polycarbonate resin base plate and fluoropolymer resin film. In addition to a reaction space, each of the containers has a liquid feed space that corresponds to the reaction space, and except for the difference of removing air when feeding the liquid, and sealing the fine groove section around the reaction space afterwards, the “a container” is the container shown in FIG. 6, the “b container” is the container shown in FIG. 20 and the “c container” is the container shown in FIG. 22. The a, b and c reaction spaces differ from that of the 8-well container in that there are no stepped sections on the top surface of the concave sections, and they are treated by hydrophilic processing instead of hydrophobic processing.

The grooves that connect to each of the spaces are 50 μm deep and 200 μm wide, and they are all treated by hydrophilic processing. All of the spaces other than the reaction spaces are constructed such that they are sealed by pressing down on cylindrical rubber seal stoppers and are raised up from the substrate surface.

The stretchable film that is applied to the bottom side is 100 μm fluoropolymer resin film, and the top blocking base that is attached to the top is a 1 mm thick polycarbonate substrate.

(Flat Container)

The “flat container” is the reaction container that is used in the second embodiment as shown in FIG. 30.

The flat container has four 0.2 mm thick circular concave sections that are formed in a 0.6 mm thick square flat plate, with two 0.1 mm deep circular stepped sections that are formed on the top end surface by injecting 90 hardness black urethane resin in a vacuum. The surface is coated with a reflective metallic film, after which hydrophobic processing is performed on the entire concave section. After each concave section is filled with liquid reaction mixture, a 1 mm thick transparent polycarbonate resin flat plate is adhered to the concave section, and with the black urethane side as the heat transfer surface and the transparent polycarbonate side as the fluorescent light measurement side, the container is submitted for the reaction.

(Flow Channel Container)

The “flow channel container” is the container used in the third embodiment as shown in FIG. 32.

The flow channel container is an approximately 2.5 mm thick rectangular flat plate.

The pattern of the flow channel is as shown in FIG. 32B, having an entire width of 200 μm and depth of 50 μm. However, the 10 mm portion of the flow channel that connects the liquid arrival observation window 2 with the PCR flow channel is greatly restricted to a width and depth of 10 μm, and the depth and width of the PCR portion is 50 μm. In this embodiment, this is the shape for pressurizing the inside of the flow channel with the back pressure of the liquid flow by applying pressure at the flow channel outlet. This 2.5 mm molded part having a groove shape is formed by injection molding of transparent polycarbonate resin. When doing this, parts for copper wiring are inserted and formed in a die, and the necessary wiring portions are formed at the same time as the injection molding. Furthermore, 10 μm thick polycarbonate film is applied to the flow channel groove side, and the container is submitted for reaction.

(Fixed Container)

The fixed container is the reaction container used in the fourth embodiment.

First, there is one circular concave section having a bottom thickness of 0.2 mm in the center of a 0.6 mm thick square flat plate, and two circumferential stepped sections having a depth of 0.1 mm are formed on the top surface around the circumference thereof by injection molding of transparent polycarbonate resin. The surface is coated with a reflective metallic film, and hydrophobic processing is performed for the entire concave section. After the liquid reaction mixture is filled into the concave section, a 100 μm thick fluoropolymer resin film is adhered to the concave section and the container is submitted for reaction.

The series of explanations related to the stable contact between container and heat source given above were described from the standpoint of having the reaction section on the substrate of the container flexibly protruding from the base plate in order to obtain stable contact between reaction section and the heat source. However, from the same viewpoint, the exact same effect can be obtained from other methods that do not center on the cushion characteristic of the container, and that will be explained below.

Even without the reaction section protruding from the container substrate, by having the heat source itself have a cushion characteristic, the same effect can be obtained without having the reaction section expand out. Therefore, the inventors covered the surface of the fixed heat sources with a heat-proof elastomer, and using a polycarbonate substrate having 8 holes as the container, PCR was performed with the reaction wells sealed in a non-stretchable 0.3 mm metallic plate instead of a stretchable film. As a result, stable nucleic acid amplification was achieved for all of the 8 wells, so it was shown that maintaining a cushion characteristic of the heat source contributes to a stable reaction. From the aspect of being heat-proof, fluoropolymer rubber or silicone rubber are suitable as the elastomer for covering the heat source.

Moreover, since the fixed heat source has a cushion, when a container is created whose metallic substrate itself is caused to be depressed, and the surface thereof is covered with film, not only the film side, but the bottom of the metallic container as well is brought into contact with the heat source, which makes even better heat transfer possible.

### Example

(Liquid Reaction Mixture)

The six types of liquid reaction mixtures shown in Table 1 are submitted for reaction.

TABLE 1

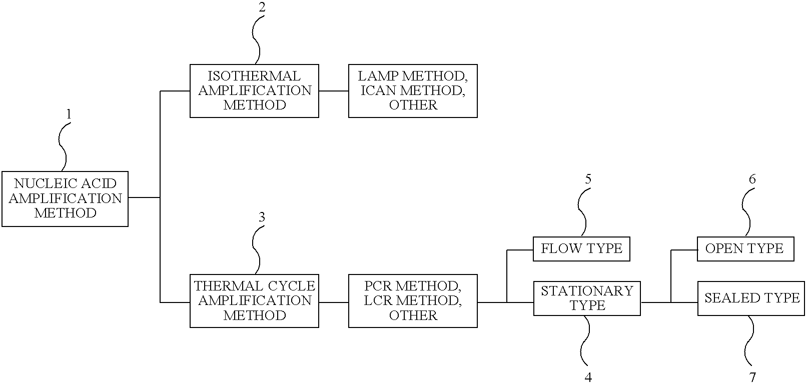

There is an offset between the target temperature required for reaction and the actual temperature at which the heat source is set, so in the actual test below, the notation target reaction temperature (set heat source temperature) is used. For example, when a reaction is to be carried out at 95° C. and the reaction is performed by setting the heat source to 110° C., the notation 95° C. (110° C.) is used.

[Experiment 1]

Liquid reaction mixture 1 was put into an 8-well container, and a PCR reaction was performed using a circular disk device (first embodiment). Thirty PCR cycles were performed at two temperatures; 0.5 seconds at the high-temperature heat source 95° C. (118° C.), and 0.5 seconds at the low-temperature heat source 55° C. (36° C.). The total reaction time, including the container rotation time, was 1 minute 6 seconds. The average output voltage of the 8 wells for real-time fluorescent light detection using a photomultiplier tube was 2.95 mV before the thermal cycles, and 3.42 mV after the reaction, so it could be confirmed that nucleic acid amplification was being performed properly.

[Experiment 2]

Liquid reaction mixture 3 was put into an 8-well container, and a PCR reaction was performed using a circular plate device from reverse transcription until detection. The reverse transcription reaction was performed for 2 minutes at the medium-temperature heat source 37° C. (37° C.). Next, the reverse transcriptase enzyme was deactivated by remaining at the high-temperature heat source 95° C. (100° C.) for 30 seconds. Then 30 PCR cycles were performed at three temperatures; 0.5 seconds at the low-temperature heat source 55° C. (36° C.), 3 seconds at the medium-temperature heat source 72° C. (77° C.), and 0.5 seconds at the high-temperature heat source 95° C. (118° C.). The total reaction time, including the container rotation time, was 5 minutes 25 seconds. The nucleic acid amplification PCR alone was 2 minutes 54 seconds. The average output voltage of the 8 wells for real-time fluorescent light detection using a photomultiplier tube was 3.11 mV before the thermal cycles, and 3.68 mV after the reaction, so it could be confirmed that nucleic acid amplification was being performed properly.

[Experiment 3]

Liquid reaction mixture 2 was put into an 8-well container and after performing a PCR reaction using a circular plate device, the amplified matter was identified by plotting a melting curve. Thirty PCR cycles were performed at three temperatures; 0.5 seconds at the high-temperature heat source 95° C. (118° C.), 3.5 seconds at the low-temperature heat source 55° C. (50° C.), and 1.5 seconds at the medium-temperature heat source 72° C. (77° C.). Here, the reason for the setting of extending the time at the low-temperature heat source and reducing the offset between the target temperature and the actual heat source temperature was in order to perform accurate heat control by reducing the amount of change in the heat source temperature when moving to the thermal offset. In order to perform melting point curve analysis of a double-helix nucleic acid, the setting of the heat source used in the last PCR cycle was different than normal. After low-temperature processing in PCR cycle 29, the temperature setting of the low-temperature heat source was changed to 55° C. After the last high-temperature processing in cycle 30, the temperature setting of the high-temperature heat source was changed to 95° C. As a result, after 30 cycles were finished, the temperature of the high-temperature heat source was 98° C., so thermal offset was performed for 1 second at the high-temperature heat source, and since the low-temperature heat source was 54° C., it was maintained for 30 seconds after which the temperature of the low-temperature heat source was raised at 0.2° C./second and fluorescent light measurement was performed one time every 2 seconds. The average value of the obtained fluorescent light of the 8 wells when plotted in a differential graph was a downward facing peak at 88.1° C. This was nearly the same as the amplified 250 by DNA separation temperature of 88.2° C., so it could be confirmed that the target amplification could be performed.

[Experiment 4]

Moreover, DNA amplification was also performed for the case in which the movement speed to each temperature heat source was changed. The reaction was performed by changing the movement speed such that movement from the medium-temperature heat source to the high-temperature heat source took 0.8 seconds, from the high-temperature heat source to the low-temperature heat source took 1 second, and from the low-temperature heat source to the medium-temperature heat source took 0.9 seconds. In this reaction as well, it could be confirmed through electrophoresis that the DNA amplification of the current and former wells were completely equivalent.

[Experiment 5]

Liquid reaction mixture 5 was put into an 8-well container, and emulsion PCR was performed. Thirty cycles were performed at three temperatures; 0.5 seconds at the high-temperature heat source 95° C. (118° C.), 0.5 seconds at the low-temperature heat source 55° C. (36° C.), and 0.5 seconds at the medium-temperature heat source 72° C. (77° C.); then after the reaction, the DNA was extracted from the emulsion, and by performing electrophoresis, it was confirmed that the target nucleic acid amplification was successful.

[Experiment 6]

Liquid reaction mixture 2 was put into the liquid feed space of the “a” container, and after feeding and sealing the liquid, a PCR reaction was performed. After putting 15 μl of the liquid reaction mixture into the liquid feed space, the seal stopper for the liquid feed space was pressed down and the container was set as was into the circular plate device, after which the PCR reaction was performed under the following conditions. Thirty cycles were performed at three temperatures; 0.5 seconds at the high-temperature heat source 95° C. (118° C.), 0.5 seconds at the low-temperature heat source 55° C. (36° C.), and 1.5 seconds at the medium-temperature heat source 72° C. (77° C.). The voltage before and after the reaction increased by 0.52 mV and the target amplification was confirmed using a photomultiplier tube.

[Experiment 7]

Liquid reaction mixture 2 was put into the liquid feed space of the “b” container, and after feeding and sealing the liquid, a PCR reaction was performed. After putting 15 μl of the liquid reaction mixture into the liquid feed space, the seal stopper for the liquid feed space was pressed down as well as the seal stopper for the atmospheric pressure release space was pressed down, then the container was set as was into the circular plate device and the PCR reaction was performed under the following conditions. Thirty cycles were performed at three temperatures; 0.5 seconds at the high-temperature heat source 95° C. (118° C.), 0.5 seconds at the low-temperature heat source 55° C. (36° C.), and 1.5 seconds at the medium-temperature heat source 72° C. (77° C.). The voltage before and after the reaction increased by 0.51 mV, so the target amplification was confirmed using a photomultiplier tube.

[Experiment 8]

Liquid reaction mixture 2 was put into the “b” container, and after feeding and sealing the liquid, a PCR reaction was performed. After putting 15 μl of the liquid reaction mixture into the liquid feed space, the seal stopper for the liquid feed space was pressed down as well as the seal stopper for the atmospheric pressure release space was pressed down. Next, silicone grease was put into the seal space and the seal stopper for the seal space was pressed to the bottom, after which the container was set as was in the circular plate device and the PCR reaction was performed under the following conditions. Thirty cycles were performed at three temperatures; 0.5 seconds at the high-temperature heat source 95° C. (118° C.), 0.5 seconds at the low-temperature heat source 55° C. (36° C.), and 1.5 seconds at the medium-temperature heat source 72° C. (77° C.). The voltage before and after the reaction was found using a photomultiplier tube to have increased by 0.47 mV, so the target amplification was confirmed.

[Experiment 9]

Liquid reaction mixture 1 was put into the “entire circumference” container, and a PCR reaction was performed using a circular plate device. In the single-direction continuous mode, 30 cycles were performed at three temperatures; high-temperature heat source 95° C. (118° C.), low-temperature heat source 55° C. (36° C.), and medium-temperature heat source 72° C. (77° C.), with one cycle being 3.3 seconds. The total reaction time was 1 minute 39 seconds. The average increase in voltage from before and after the reaction according to a photomultiplier tube for the 60 reaction spaces was 0.33 mV with a standard deviation of 0.03 mV, so it was confirmed that nucleic acid amplification was performed well for all of the reaction spaces.

[Experiment 10]

Liquid reaction mixture 2 was put into a flat plate container and a PCR reaction was performed using a flat plate device (second embodiment). Thirty cycles were performed at three temperatures; 2 seconds at the high-temperature heat source 95° C. (102° C.), 2 seconds at the low-temperature heat source 55° C. (43° C.), and 2 seconds at the medium-temperature heat source 72° C. (76° C.). The total reaction time was 3 minutes 59 seconds. The increase in voltage from before and after the reaction was found according to a photomultiplier tube to be 0.54 mV, so the target amplification was confirmed.

[Experiment 11]

Liquid reaction mixture 4 was used in a flow channel device (third embodiment) and a reaction was performed from reverse transcription to electrophoresis. The flow channel container was set in the flow channel device, after which liquid reaction mixture 4 was put into a special syringe that was then inserted into the liquid inlet of the flow channel container and the reaction was started. The liquid feed speed during the reaction was 0.012 μl/sec. A reverse transcription reaction was performed at a reverse transcription heat source 37° C. (37° C.). The calculated elapsed time was 2 minutes. Next, the reverse transcriptase enzyme deactivation heat source was 95° C. (100° C.), and the calculated elapsed time was 30 seconds. For 30 cycles at a low-temperature heat source 55° C. (51° C.) and high-temperature heat source 95° C. (91° C.), the PCR unit required a passage time of approximately 4 minutes. The flow channel container was removed after 6 minutes 40 seconds, after which Agarose gel for electrophoresis was injected from the electrophoresis liquid inlet and fed until it reached the liquid arrival observation opening on the electrophoresis side. After that, the container was set again in the flow channel device, the electrophoresis start button was pressed and electrophoresis was performed. During this time, a state was observed by way of the reflected light measurement probe and spectrometer that were inserted into the fluorescent light measurement unit in which the primer dimer that was created at the same time as the 208 by band of the G3PDH area was separated.

[Experiment 12]

Liquid reaction mixture 6 was put into a fixed container (fourth embodiment) and sealed, after which a LAMP reaction was performed. After keeping the container at a temperature of 65° C. for one hour, it was visually observed that the liquid was cloudy, which indicated that target reaction was progressing stably.

(Kit)

A kit for the purpose of detecting MRSA (Methicillin-resistant Staphylococcus Aureus) was created having the following composition.

A user uses a circular plate device and performs real-time PCR using a polymerase and buffer that can be provided by the user.

Five 8-well containers

20 ml of primer MIX (F primer array: AACTGTTGGCCACTATGAGT, R primer array: CCAGCATTACCTGTAATCTCG)

What is claimed is:

1. A reaction promoting device for providing, in a container having a reaction part holding a reaction solution, the reaction solution with a predetermined thermal cycle thereby promoting a thermal cycle reaction such as PCR(polymerase chain reaction), LCR (ligase chain reaction), or the like comprising:
a thermal cycle heater which is disposed to face a reaction part of the container and provides the thermal cycle by controlling the temperatures of the reaction solution held in the container, within a predetermined thermal cycle time, to a high-temperature-side target temperature, a medium-temperature-target temperature, and a low-temperature-side target temperature; and
a temperature controller part that controls the temperatures of the thermal cycle so as to offset the high-temperature-side thereof, from the high-temperature-side target temperature, by not less than a predetermined temperature difference, and so as to offset the low-temperature-side thereof, from the low-temperature-side target temperature, by not less than a predetermined temperature difference.

2. The reaction promoting device as set forth in claim 1, wherein the device is so controlled that the temperature offset is equal to or greater than a temperature difference defined by the equation below:
Y=5,000X³−900X²+50X+2.8 (a)
where X=Depth (mm) of the reaction solution×thickness (mm) of a container heat transfer surface, wherein the container heat transfer surface is a film portion sandwiched between a surface of a container flat part in contact with a solid heat source and the reaction solution.

3. The reaction promoting device as set forth in claim 1, wherein the thermal cycle heater comprises
a high temperature part, a medium temperature part, and low temperature part; and
a drive part which provides a predetermined thermal cycle to the reaction solution held in the reaction part by displacing a relative position of the thermal cycle heater with respect to the container, wherein
the drive part controls a period of time during which the container faces the heater by coordinating the period of time with the predetermined thermal cycle time.

4. The reaction promoting device as set forth in claim 3, wherein
the high temperature part, medium temperature part, and low temperature part are disposed in a circumferential direction;
the drive part drives the container or the heater so that the relative position of the container with respect to the heater is displaced in a rotational circumferential direction.

5. The reaction promoting device as set forth in claim 4, wherein
the drive part intermittently causes a relative displacement of the thermal cycle heater and the reaction container while they are in contact with each other, thereby causing the reaction solution held in the container to reside for a predetermined time in the thermal heater's high temperature part, medium temperature part, and low temperature part, respectively.

6. The reaction promoting device as set forth in claim 5, wherein
the control part adjusts a speed with which a specific part on the container moves until the specific part faces the low temperature part of the thermal cycle heater, a speed with which the specific part moves from the low temperature part to the medium temperature part, and a speed with which the specific part exits from the medium temperature part, thereby holding a thermal history applied to the specific part constant at least in regard to the low and medium temperatures.

7. The reaction promoting device as set forth in claim 4, wherein
the drive part continuously causes a relative displacement of the thermal cycle heater and the reaction container while they are in contact with each other.

8. The reaction promoting device as set forth in claim 3, wherein
the high temperature part, medium temperature part, and low temperature part are disposed along a linear direction, and
the drive part drives the container or the heater so that the relative position of the container with respect to the heater is displaced in a linear direction.

9. The reaction promoting device as set forth in claim 3, wherein
the control part drives, when displacing the thermal cycle heater from the reaction container, to separate the cycle heater from the reaction container and displace them while separated, and thereafter to bring the cycle heater into contact with the reaction container.

10. The reaction promoting device as set forth in claim 1, wherein
the device further comprises an optical detector apparatus for reading out how well the reaction has progressed; and
the container has at least part thereof formed of a light shielding material or is tinted to shield light for preventing an optical interference of a plurality of the reaction solutions.

11. The reaction promoting device as set forth in claim 10, wherein
the container uses an optically transparent pressing member as a member to press the container; and an optical measurement is made through the transparent pressing member.

12. The reaction promoting device as set forth in claim 1, wherein
the device has an apparatus that presses the container flat plate on its front and back faces;
at least one side of the pressing apparatus structure is a solid heat source;
at least one side of the reaction part of the flat part is a 10-400 μm thick film heat transfer surface; and
the solid heat source is allowed to face the film heat transfer surface and is brought into contact therewith by the pressing apparatus, thereby heating the reaction solution and a rise in an internal pressure of the reaction part causes the film heat transfer surface to expand, thereby securing a stable contact of the container with the heat source.

13. The reaction promoting device as set forth in claim 1, wherein
the container is a reaction container that has a flat plate part wherein a concave part is formed into the flat plate part thereof; a reaction solution, which is an aqueous solution, is placed in the concave part; thereafter the concave part is sealed, thereby sealing off the reaction solution; and a solid heat source set at a temperature corresponding to the temperature needed for the reaction is brought into contact with the flat plate part thereby performing nucleic acid amplification reaction, a thermal reaction; and wherein
at least one circumferential lip is formed on an upper end face of the concave part; and both the circumferential lip part and the lower face of the seal member are made hydrophobic, thereby allowing the nucleic acid amplification reaction to be stably performed.

14. The reaction promoting device as set forth in claim 1, wherein
the container further comprises
a liquid inlet aperture with an open end for introducing a reaction solution under pressure into a reaction part, which aperture is connected to the reaction part via a liquid transport groove-shaped aperture in the container; and
a mechanism that allows introducing the reaction solution through the open end of the liquid inlet aperture, then transporting the reaction solution to the aperture for reaction through the liquid transport groove-shaped aperture, thereafter sealing the open end with a seal stopper, and
pressing to permit a reaction to occur while the seal stopper is held under pressure so as not to cause the seal stopper to come loose during the reaction.

15. The reaction promoting device as set forth in claim 14, wherein
the seal stopper comprises an air groove extending from the middle part of the seal stopper toward the lower part thereof.

16. The reaction promoting device as set forth in claim 14, wherein
the container comprises
an air vent groove-shaped aperture connected to the reaction part;
a seal liquid aperture filled with a seal liquid for allowing the seal liquid to flow; and
a groove-shaped aperture that connects the seal liquid aperture to the air vent groove-shaped aperture and the liquid inlet aperture, and wherein
after transport of the reaction solution from the liquid inlet aperture to an aperture for reaction, the seal liquid is transported from the aperture filled with the seal liquid, thereby introducing the seal liquid into both the groove-shaped aperture on the liquid inlet aperture side of the aperture for reaction and the groove-shaped aperture on the air vent side groove-shaped aperture.

17. The reaction promoting device as set forth in claim 16, wherein the seal liquid is composed of a high viscosity grease-like liquid including silicone grease, or a UV curable resin, or a thermoset resin.

18. The reaction promoting device as set forth in claim 1, wherein
the device, in which a part holding a reaction solution in a flat part is flow channel-shaped, is for performing a flow type nucleic acid amplification reaction that runs amplification reaction while the reaction solution is allowed to flow, further comprising
a liquid transport controller for controlling the rate at which the reaction solution is transported.
